(19) United States
(12) Patent Application Publication
(10) Pub. No.: US 2015/0034082 A1
(43) Pub. Date: Feb. 5, 2015

(54) OXYGENATION-VENTILATION METHODS AND SYSTEMS

(71) Applicant: Covidien LP, Mansfield, MA (US)

(72) Inventors: Gardner Kimm, Carlsbad, CA (US); Mehdi Jafari, Laguna Hills, CA (US); Warren Sanborn, Escondido, CA (US)

(21) Appl. No.: 13/958,769

(22) Filed: Aug. 5, 2013

(51) Int. Cl. A61M 16/00; A61B 5/00; A61B 5/145; A61B 5/0205; A61B 5/026

(52) U.S. Cl. CPC A61M 16/0051; A61M 16/0069; A61M 16/0003; A61B 5/0205; A61B 5/026; A61B 5/14539; A61B 5/14542; A61B 5/4836; A61B 5/091
USPC 128/202.16

(57) ABSTRACT

The systems and methods include providing oxygenation-ventilation of a patient being monitored or treated with a medical system. The novel systems and methods may be configured to be able to simultaneously treat and/or monitor a patient with both a ventilator and an ECGE system. The novel systems and methods may monitor the patient and/or manage the treatment of a patient with a ventilator-ECGE system.

### INTRODUCTION

Medical ventilator systems have long been used to provide supplemental to total ventilatory support to patients. These ventilators typically comprise a source of pressurized air and oxygen which is fluidly connected to the patient through a conduit. Some ventilators have been adapted to monitor the patient to ensure that the patient is being properly ventilated.

Extracorporeal membrane gas-exchange (ECGE) devices or more commonly known as lung dialysis machines provide life-saving temporary lung support to a patient who experiences respiratory failure. ECGEs or lung dialysis machines are utilized when patients are unresponsive or failing under standard ventilator management. ECGEs or lung dialysis machines remove carbon dioxide from a patient's blood and oxygenate a patient's blood using intrathoracic or extrathoracic cannulation.

### Oxygenation-Ventilation Methods and Systems

This disclosure describes systems and methods for oxygenation-ventilation of a patient being monitored or treated by a medical system, such as a ventilator-extracorporeal membrane gas-exchange (ECGE) system. The disclosure describes novel systems and methods for treating and/or monitoring a patient with a medical system, such as a ventilator-ECGE system. The disclosure also describes novel systems and methods for monitoring the patient and/or managing the treatment of a patient with a medical system, such as a ventilator-ECGE system.

In part, this disclosure describes a method for managing the treatment of a patient connected to a ventilator-ECGE system. The method including:
delivering breathing gas to a patient with a ventilator system based on one or more ventilator settings;
monitoring ventilator sensors with the ventilator system;
removing carbon dioxide from and adding a gas mixture to a bloodstream of the patient to oxygenate the bloodstream of the patient with an extracorporeal membrane gas-exchange (ECGE) system based on one or more ECGE settings;
monitoring ECGE sensors with the ECGE system;
communicating information between the ventilator system and the ECGE system;
comparing sensor output from the ventilator sensors to a first threshold;
comparing the sensor output from the ECGE sensors to a second threshold;
adjusting at least one of the one or more ventilator settings and the one or more ECGE settings based on the comparing of the sensor output to the first threshold and the comparing of the sensor output to the second threshold.

Yet another aspect of this disclosure describes a ventilator-ECGE system including at least one processor and at least one memory. The at least one memory is communicatively coupled to the at least one processor. The memory contains instructions that, when executed by the at least one processor cause a controller to issue a notification. The controller includes a manage module, a notification module, and a display module. The manage module detects improper treatment of a patient based on sensor output from a ventilator sensor and/or from an ECGE sensor. The improper treatment includes detecting at least one of improper oxygenation and/or improper ventilation. The notification module determines an appropriate message based on the improper treatment and determines an appropriate entry based on the improper treatment. The display module issues at least one of the appropriate message and the appropriate entry.

The disclosure further describes a ventilator-ECGE system including a ventilator system, an ECGE system, an operator interface, a plurality of sensors, a controller, and a display module. The ventilator system includes a pressure generating system. The pressure generating system is adapted to deliver breathing gas to a patient based on ventilator settings. The ECGE system removes carbon dioxide from a patient's bloodstream and provides a gas mixture to the patient's bloodstream to oxygenate the patient's bloodstream based on ECGE settings. The operator interface receives operator input. The plurality of sensors are operatively coupled to at least one of the pressure generating system, the patient, a patient circuit, and the patient's bloodstream. The plurality of sensors monitors a plurality of parameters to generate sensor output. The controller receives the operator input and commands the pressure generating system and the ECGE system based on the operator input. The display module displays the generated sensor output.

Yet another aspect of this disclosure describes a method for managing treatment of a patient connected to medical system. The method includes:
delivering breathing gas to a patient with a ventilator system based on one or more ventilator settings;
monitoring ventilator sensors with the ventilator system;
monitoring oximeter sensors with an oximeter;
monitoring capnometer sensors with a capnometer;
removing carbon dioxide from and adding a gas mixture to a bloodstream of the patient to oxygenate the bloodstream of the patient with an extracorporeal membrane gas-exchanger (ECGE) system based on one or more ECGE settings;
monitoring ECGE sensors with the ECGE system;
communicating information between the ventilator system, the capnometer, the oximeter, and the ECGE system;
receiving a desired threshold;
comparing the desired threshold to sensor output from at least one of the capnometer sensor and the oximeter sensor; and
adjusting the one or more ventilator settings to maintain the desired ventilation threshold based on the comparing of the desired threshold to the sensor output.

The disclosure further describes a method for optimizing synchronization of heart and lung movements in a patient with a ventilator-ECGE system. The method includes:
delivering breathing gas to a patient with a ventilator system based on one or more ventilator settings;
removing carbon dioxide from and adding a gas mixture to a bloodstream of the patient to oxygenate the bloodstream of the patient with an arteriovenous extracorporeal membrane gas-exchanger (ECGE) system based on one or more ECGE settings,
wherein the arteriovenous ECGE system increases a cardiac load of the patient;
detecting the timing of cardiac systolic and diastolic cycles;
synchronizing the triggering of inspiration and exhalation of the patient with the ventilator system based on the detected timing to offset a portion of the increased cardiac load caused by the arteriovenous ECGE.

Additionally, the disclosure further describes a ventilator-ECGE system including a ventilator system, an ECGE system, an operator interface, a capnometer, an oximeter, a plurality of sensors, a controller, and a display module. The ventilator system includes a pressure generating system. The pressure generating system is adapted to deliver breathing gas to a patient based on ventilator settings. The ECGE system removes carbon dioxide from a patient's bloodstream and provides a gas mixture to the patient's bloodstream to oxygenate the patient's bloodstream based on ECGE settings. The operator interface receives operator input. The plurality of sensors are operatively coupled to at least one of the pressure generating system, the patient, a patient circuit, and the patient's bloodstream. The plurality of sensors monitors a plurality of parameters to generate sensor output. The controller receives the operator input and commands the pressure generating system and the ECGE system based on the operator input. The display module displays the generated sensor output.

Additionally, the disclosure further describes a ventilator-ECGE system including a ventilator system, an arteriovenous ECGE system, a synchronization module, and a plurality of sensors. The ventilator system includes a pressure generating system. The pressure generating system is adapted to deliver breathing gas to a patient based on ventilator settings. The arteriovenous ECGE system removes carbon dioxide from a patient's bloodstream and provides a gas mixture to the patient's bloodstream to oxygenate the patient's bloodstream based on arteriovenous ECGE settings. The plurality of sensors are operatively coupled to at least one of the pressure generating system, the patient, a patient circuit, and the patient's bloodstream. The plurality of sensors monitors a plurality of parameters to generate sensor output. The synchronization module triggers inspiration and/or exhalation of the patient with the ventilator system based on detected timing of cardiac systolic and diastolic cycles from sensor output to offset a portion of the increased cardiac load caused by the arteriovenous ECGE device.

These and various other features as well as advantages which characterize the systems and methods described herein will be apparent from a reading of the following detailed description and a review of the associated drawings. Additional features are set forth in the description which follows, and in part will be apparent from the description, or may be learned by practice of the technology. The benefits and features of the technology will be realized and attained by the structure particularly pointed out in the written description and claims hereof as well as the appended drawings.

It is to be understood that both the foregoing general description and the following detailed description are exemplary and explanatory and are intended to provide further explanation of the disclosure and the claims.

### BRIEF DESCRIPTION OF THE DRAWINGS

The following drawing figures, which form a part of this application, are illustrative of embodiments, systems, and methods described below and are not meant to limit the scope of the disclosure in any manner, which scope shall be based on the claims appended hereto.

FIG. 1 illustrates an embodiment of a ventilator-extracorporeal membrane gas-exchanger system connected to a human patient.

FIG. 2 illustrates an embodiment of a ventilator-extracorporeal membrane gas-exchanger system connected to a human patient.

FIG. 3 illustrates an embodiment of a ventilator-extracorporeal membrane gas-exchanger system connected to a human patient.

FIG. 4 illustrates an embodiment of a ventilator-extracorporeal membrane gas-exchanger system connected to a human patient.

FIG. 5 illustrates an embodiment of a method for managing the treatment of a patient connected to a medical system.

FIG. 6 illustrates an embodiment of a method for issuing a notification upon detecting an implication of improper patient treatment with a ventilator-extracorporeal membrane gas-exchanger system.

FIG. 7 illustrates an embodiment of a method for optimizing synchronization of the heart and lung movements in a patient being treated with a ventilator-ECGE system.

FIG. 8 illustrates an embodiment of a graphical user interface displaying data from a ventilator and an ECGE system.

FIG. 9 illustrates an embodiment of a graphical user interface displaying a selectable drop down menu for a ventilator-ECGE system.

FIG. 10 illustrates an embodiment of a graphical user interface displaying a notification including a message and a recommendation entry for a ventilator-ECGE system.

FIG. 11 illustrates an embodiment of a method for managing the treatment of a patient connected to a medical system.

### DETAILED DESCRIPTION

Although the techniques introduced above and discussed in detail below may be implemented for a variety of medical devices, the present disclosure will discuss the implementation of these techniques in the context of a ventilator-extracorporeal membrane gas-exchanger (ECGE) system for use in providing oxygenation-carbon dioxide (PₐO₂ and PₐCO₂) management for a human patient. A person of skill in the art will understand that the technology described in the context of a medical ventilator-ECGE system for human patients could be adapted for use with other systems such as systems for non-human patients and general gas transport systems.

The metabolic homeostasis of a patient depends on the body's ability to maintain PₐO₂, PₐCO₂ and pH within narrow limits. In the intensive care unit approximately 40% of patients are placed on ventilators for the purpose of assisting the patient's respiratory function or completely substituting for the patient's respiratory function (e.g., when the patient is paralyzed). For some patients, however, the loss of lung function is so severe that even the mildest form of ventilatory support is at best futile and at worst likely to exacerbate any existing lung injury, which could lead to or hasten the death of the patient. For example, ventilation of some patients, such as patients suffering acute respiratory distress syndrome (ARDS), acute lung injury (ALI) or ventilator induced lung injury (VILI), often requires the use of a high driving pressure to open up stiff lungs. However, the high driving pressure necessary to ventilate lungs may also damage the lungs.

Another type of biomechanical device, an extracorporeal membrane gas-exchanger (ECGE) system, has the capacity to substitute in part or totally for the failing or failed lung. The ECGE system has the capability of providing, ex-vivo, gas exchange for the failing lung. However, the ECGE system is very invasive, requires the removal of blood from the patient, and increase the risk of infection. If the ECGE system is utilized too long or too intensely, the ECGE system can lead to the necrosis of a patient limb or distal limb ischemia. Also, the removal of the blood can increase the risk of infection compared to patients that do not require the removal of any blood. Accordingly, the ECGE system is only utilized when necessary. Further, arteriovenous ECGE systems increase the load on the patient's cardiac system and can only be utilized in patients with a cardiac system strong enough to maintain the extra cardiac load created by use of the arteriovenous ECGE system. Also, during 100% ECGE support (and the absence of tidal-lung ventilation), the lung will become atelectatic and therefore prone to infection and the diaphragm will become quiescent and therefore prone to atrophy and decrease the chances of the patient ever being able to breathe independently. Moreover, if there is a leak in the ECGE system, the patient risks death from exsanguination. Hence, an ECGE system exposes the patient to additional risk, but is often the only method left for oxygenating and carbon dioxide removal for a patient when use of a mechanical ventilator is more damaging than beneficial.

ECGE as used herein is a general byword for the wide range of methods that are used for extracorporeal blood oxygenation and carbon dioxide removal. Several different terms have been used for these systems to describe the wide range of methods that are used for extracorporeal blood oxygenation and carbon dioxide removal. Accordingly, the ECGE system as used herein includes the following artificial cardiac and/or pulmonary support systems and terms: extracorporeal membrane oxygenation (ECMO); lung dialysis; extracorporeal life support (ECLS); extracorporeal carbon dioxide removal (ECCOR or ECCO₂R); partial extracorporeal carbon dioxide removal (PECCO₂); Arteriovenous carbon dioxide removal (AVCOR), extracorporeal lung assist (ECLA); extracorporeal gas-exchanger; and intravascular oxygenator (IVOX). The list provided above is exemplary only and is not meant to be limiting Many of these systems perform their oxygenation and carbon-dioxide removal functions in similar or different ways. As used herein “ECGE” or an “ECGE system” refers to any suitable lung dialysis system for performing blood oxygenation and carbon dioxide removal.

Accordingly, the present disclosure describes systems and methods for managing the oxygenation and carbon dioxide removal (tissue/organ PₐO₂ and PₐCO₂) of a patient with a ventilator-ECGE system.

In the management of the O₂—CO₂-gas-exchange function of the failing lung, it must be recognized that neither the ventilator, operating alone as the primary means of ventilatory support nor the extracorporeal membrane gas-exchanger, operating alone as the primary means of ventilatory support, is a curative device. Rather, the aim of the combination system is to augment, either partially or totally, the gas-exchange function of the compromised lung, whether through the cooperative interaction of both systems or through the individual action of one single system. What this invention proposes is that if failing gas exchange is of critical significance in the management of a patient, the combined integration of a ventilator system and an extracorporeal membrane gas-exchanger system into an intelligent control and management gas-exchange system will have greater advantages than either separate system acting alone.

For example, a patient who requires an ECGE system for proper oxygenation may also be ventilated at a lower level that would not cause injury to the patient's lung. This reduced level of ventilation might mitigate the probability of atelectasis and the concurrent development of diaphragm atrophy while at the same time providing enough oxygenation/ventilation to the patient to allow the ECGE system to operate at a reduced blood flow. The lower blood flow rate may increase the time the ECGE system can be utilized without causing necrosis or prevent necrosis from happening. Further, the ventilator can be operated to relieve cardiac load and may be utilized to offset the increased cardiac load of an arteriovenous ECGE systems. Accordingly, the ventilator-ECGE system allows patients who previously would not have been able to use an ECGE system because of the increased cardiac load to use an ECGE system in combination with mechanical ventilation.

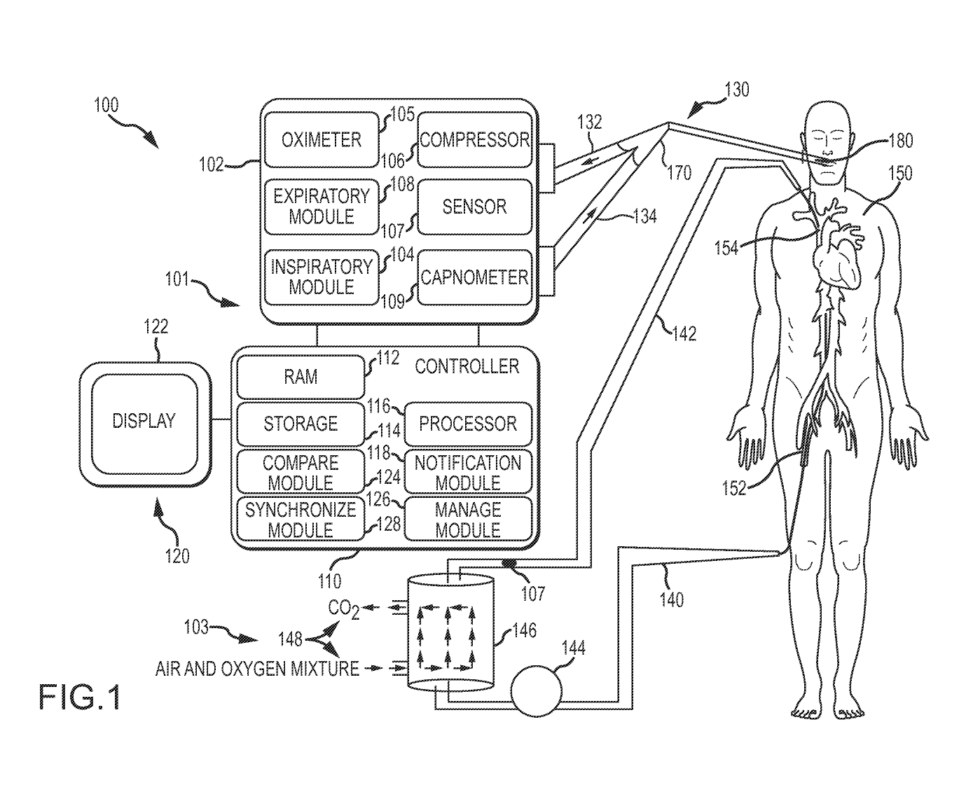

FIGS. 1-4 are schematic diagrams illustrating an embodiment of an exemplary ventilator-ECGE system 100 connected to a human patient 150. The ventilator-ECGE system 100 includes a ventilator 101 and an ECGE system 103 that communicates data between each other. For example, the ECGE system 103 may send information to the ventilator 101, the ventilator 101 may send information to the ECGE system 103, or the ventilator 101 and the ECGE system 103 may send information back and forth to each other. This communication allows the ventilator 101 and/or ECGE 103 to send data, instructions, and/or commands to each other. The ventilator 101 and ECGE system 103 may be electrically or physically connected to each other. In some embodiments, the ventilator 101 and the ECGE system 103 are wirelessly connected to each other. In some embodiments, a cable connects the ventilator 101 and the ECGE system 103 to each other. In other embodiments, the ventilator 101 and the ECGE system 103 are physically connected to each other. For example, the ventilator 101 and the ECGE system 103 may share a portion of the same housing or enclosure.

The ventilator-ECGE system 100 treats the patient 150 by utilizing the ventilator 101 and/or the ECGE system 103. Therefore, in some embodiments, the ventilator-ECGE system 100 utilizes the ventilator 101 and the ECGE system 103 concurrently, overlapping in time, simultaneously, and/or individually at separate times. The treatment provided by the ventilator-ECGE system 100 may be adjusted and dynamically changed based on the patient 150, generated sensor output, and/or selected settings. Accordingly, a patient 150 being treated with both the ventilator 101 and the ECGE system 103 may after a period of treatment be treated with just the ventilator 101 or the ECGE system 103. In another example, a patient 150 being treated with the ECGE system 103 after a period of time may be treated with both the ventilator 101 and the ECGE system 103. In some embodiments, the ventilator-ECGE system 100 uses the ventilator 101 and/or the ECGE system 103 to treat the patient 150 based on operator input. In other embodiments, the ventilator-ECGE system 100 determines to use the ventilator 101 and/or the ECGE system 103 to treat the patient 150 based on predetermined settings and patient characteristics, such as patient age, patient height, patient weight, patient gender, patient disease state, ventilation mode, and/or sweep gas flow rate. This list is exemplary only and is not meant to be limiting.

Ventilator 101 includes a pneumatic gas delivery system 102 (also referred to as a pressure generating system 102 or pneumatic system 102) for circulating breathing gases to and from patient 150 via the ventilation tubing system 130, which couples the patient 150 to the pneumatic system 102 via an invasive (e.g., endotracheal tube, as shown) or a non-invasive (e.g., nasal mask) patient interface 180.

Ventilation tubing system 130 (or patient circuit 130 or breathing circuit 130) may be a two-limb (shown) or a one-limb circuit for carrying gases to and from the patient 150. In a two-limb embodiment, a fitting, typically referred to as a “wye-fitting” 170, may be provided to couple a patient interface 180 (as shown, an endotracheal tube) to an inspiratory limb 134 and an expiratory limb 132 of the ventilation tubing system 130.

Pneumatic system 102 may be configured in a variety of ways. In the present example, pneumatic system 102 includes an expiratory module 108 coupled with the expiratory limb 132 and an inspiratory module 104 coupled with the inspiratory limb 134. Compressor 106 or other source(s) of pressurized gases (e.g., air, oxygen, and/or helium) is coupled with inspiratory module 104 and the expiratory module 108 to provide a gas source for ventilatory support via inspiratory limb 134.

The inspiratory module 104 is configured to deliver gases to the patient 150 according to prescribed ventilatory settings. The expiratory module 108 is configured to release gases from the patient's lungs according to prescribed ventilatory settings. Specifically, expiratory module 108 is associated with and/or controls an expiratory valve for releasing gases from the patient 150.

The ventilator 101 may also include one or more sensors 107 communicatively coupled to ventilator 101 and/or patient 150. The sensors 107 may be located in the pneumatic system 102, ventilation tubing system 130, and/or on the patient 150. The embodiment of FIG. 1 illustrates a sensor 107 in pneumatic system 102.

Sensors 107 may communicate with various components of ventilator 101, e.g., pneumatic system 102, other sensors 107, processor 116, controller 110, compare module 124, manage module 126, notification module 118 and/or any other suitable components and/or modules. Sensors 107 may communicate with various components of the ECGE system 103. Sensors 107 may communicate with both the ECGE system 103 and the ventilator 101. In one embodiment, sensors 107 generate output and send this output to pneumatic system 102, other sensors 107, processor 116, compare module 124, manage module 126, notification module 118 and/or any other suitable components and/or modules. The sensor output may be data received directly from the sensors 107 (e.g., gas concentration) or may be parameters derived from received sensor data (e.g., work of breathing, patient effort, etc.). Sensors 107 may employ any suitable sensory or derivative technique for monitoring one or more parameters associated with the patient 150 and the oxygenation and/or ventilation of a patient 150. The term “ventilation” refers to the management of carbon dioxide (such as carbon dioxide removal) when used herein. Sensors 107 may detect changes in patient parameters indicative of patient triggering, for example. Sensors 107 may be placed in any suitable location, e.g., within the ventilatory circuitry 130, blood circuit 143, or other devices communicatively coupled to the ventilator 101 and/or ECGE system 103. For example, sensors 107 may be placed in any suitable internal location, such as, within the ventilatory circuitry 130, blood circuit 143, and/or or within components or modules of ventilator 101. Any sensory device useful for monitoring changes in measurable parameters during an oxygenation and/or ventilation of the patient 150 may be employed in accordance with embodiments described herein.

As should be appreciated, ventilator sensor output and/or ECGE sensor output are highly interrelated and, according to embodiments, may be either directly or indirectly monitored. That is, parameters or variables may be directly monitored by one or more sensors 107, as described above, or may be indirectly monitored or estimated/calculated using a model, such as a model derived from the Equation of Motion (e.g., Target Airway Pressure(t)=Eₚ∫Qₚdt+QₚRₚ−Patient Effort(t)).

The pneumatic system 102 may include a variety of other components, including an oximeter 105, a capnometer 109, mixing modules, valves, tubing, accumulators, filters, and/or etc.

The ECGE system 103 of the ventilator-ECGE system 100 may be any suitable ECGE system 103 as known by a person of skill in the art. The ECGE system 103 of the ventilator-ECGE system 100 removes or shunts blood from a patient 150, removes carbon dioxide from the removed blood, adds oxygen to the removed blood, and then reinserts the oxygenated blood back into the patient 150. In other words, the ECGE system 103 of the ventilator-ECGE system 100 diverts blood from the patient for the removal of carbon dioxide and addition of oxygen before being returned to the patient.

The ECGE system 103 includes a membrane lung 146 to remove the carbon dioxide from the removed blood and to oxygenate the removed blood. An access tube 140 connects a vein or an artery of the patient 150 to the membrane lung 146. A return tube 142 connects the membrane lung 146 to a vein of the patient 150. The access tube 140, the return tube 142, and the membrane lung 146 create a blood flow path or blood circuit 143 from the patient 150 through the membrane lung 146 and back to the patient 150. In some embodiments, the access tube 140 and the return tube 142 are catheters. In some embodiments, the access tube 140 and the return tube 142 are cannula. In some embodiments, the access tube 140 and the return tube 142 are separate from each other as illustrated in FIG. 1. In other embodiments, the access tube 140 and the return tube 142 are a dual lumen or double lumen system. However, the access tube 140 and the return tube 142 may be any suitable type of tubing for removing and/or inserting blood into the patient 150 for use in an ECGE system 103.

In some embodiments, the blood circuit 143 provides a place for infusions into the blood, such as a drug infusion. For example, in some embodiments, systematic heparin administration is provided to the blood of the patient 150 through the blood circuit 143 to prevent thrombus formation within the blood circuit 143.

The ECGE system 103 utilizes a membrane lung 146 to remove the carbon dioxide from the removed blood and to oxygenate the removed blood. The membrane lung 146 of the ECGE system 103 may be any suitable membrane lung 146 for removing carbon dioxide and oxygenating a patient's blood in an ECGE system 103. For example, the membrane lung 146 may be a coiled silicone rubber membrane lung or a hollow fiber membrane. In embodiments, where a hollow fiber membrane lung 146 is utilized, the hollow fibers may be microporous polypropylene and/or nonporous poly-4-methyl-1-pentene (PMP). In some embodiments, the membrane lung 146 is coated to improve biocompatibility and/or to decrease clotting. In some embodiments, the membrane lung 146 is a Medos Hilite® as sold by Medos Medizintechnik AG, which is located at Obere Steinfurt 8-10, D-52222 Stolberg, Germany. In other embodiments, the membrane lung 146 is an iLA Membrane Ventilator as sold by Novalung, GmbH, which is located at Im Zukunftspark, 74076 Heilbronn, Germany. In other embodiments, the membrane lung 146 is a Maquet Quadrox D, as sold by MAQUET Medical Systems USA, which is located at 45 Barbour Pond Drive, Wayne, N.J. 07470. However, these membrane lungs are merely exemplary and are not meant to be limiting.

In some embodiments, the ECGE system 103 is an arteriovenous system. The arteriovenous system removes or shunts blood from an artery of the patient 150 and reinserts the removed blood into the patient 150 via a vein of the patient 150. For example, in such an arteriovenous system normally oxygenated blood is shunted via a suitable catheter from a nominally high-pressure artery through a membrane, gas-exchange device/module back to a lower-pressure vein via a suitable catheter. In some embodiments, an arteriovenous system removes blood from the right common carotid, auxiliary, femoral, or aortic artery and then reinserts the oxygenated blood back into the patient 150 via the jugular or femoral vein 152. The arteriovenous system utilizes the pressure gradient between artery and vein to push blood through the system and does not require the use of a pump.

In some embodiments, the ECGE system 103 is a venovenous system as illustrated in FIG. 1. A venovenous system removes blood from a vein of a patient 150 and reinserts the removed blood into the patient 150 via another vein of the patient 150. For example, in some embodiments, a venovenous system removes blood via the femoral vein 152 and then reinserts the blood back into the patient 150 via the internal jugular vein 154 as illustrated in FIG. 1. In other embodiments, the venovenous system removes blood via the femoral or right atrium vein and then reinserts the blood back into the patient 150 via the same vein. In other embodiments, the venovenous system removes blood via the femoral, jugular, or saphenous vein and then reinserts the blood back into the patient 150 via the femoral or saphenous vein. The venovenous system like the veno-arterial system utilizes a pump 144 to move the removed blood through the membrane lung 146 and back into the patient 150.

In other embodiments, the ECGE system 103 is a veno-arterial system. The veno-arterial system removes blood from a vein of the patient 150 and reinserts the removed blood into the patient 150 via an artery of the patient 150. For example, in some embodiments, a veno-arterial system removes blood from the femoral vein and then reinserts the oxygenated blood back into the patient 150 via the femoral artery. The veno-arterial system differs from the arteriovenous system because the veno-arterial system utilizes a pump to push blood through the system.

In some embodiments, a centrifugal pump is utilized by the ECGE system 103. In other embodiments, a roller pump or impeller is utilized by the ECGE system 103. However, any suitable pump 144 for moving blood through an ECGE system 103 may be utilized. As discussed above, the arteriovenous system does not require a pump 144 and instead utilizes the patient's own pump, the heart, to move the blood through the membrane lung 146 and to move the blood back into the patient 150. However, the cannula and membrane of the arteriovenous system creates extra length external to the patient 150 that the heart must now pump blood through, increasing the work load (i.e., an increase in resistance to flow) on the heart of the patient 150.

Blood containing carbon dioxide flows into the membrane lung 146. An input gas, such as air, is pumped into the membrane lung 146 and an exhaust gas, such as carbon dioxide is removed from the membrane lung 146. The input gas and the exhaust gas are referred to herein as the sweep gas 148. The input gas flows into the membrane of the membrane lung 146 while blood is flowing through the membrane of the membrane lung 146. As the input gas flows through the membrane containing the input gas, carbon dioxide is removed from the blood and the blood becomes oxygenated by the input gas. However, in some instances the carbon dioxide removal rate is more efficient than the oxygenation rate. The carbon dioxide removed from the blood and any components of the input air that are not absorbed into the blood, if any, exit the membrane lung 146 making the exhaust gas. The flow rate of the sweep gas 148 as the sweep gas 148 flows through the membrane lung 146 is adjustable. The speed or flow rate of the sweep gas 148 affects the rate of CO₂ removal from the patient. Further, in ECGE systems 103 that utilize a pump 144, the flow rate of the blood through the membrane lung 146 is adjustable. The speed at which the blood is pumped through the ECGE system 103 or the blood flow rate affects the oxygen exchange and arterial oxygenation of the patient.

The ECGE system 103 may also include one or more sensors 107 communicatively coupled to the ECGE system 103 and/or patient 150. In some embodiments, the sensors 107 are located in the blood circuit 143, access tube 140, return tube 142, and/or the membrane lung 146. FIG. 1 illustrates a sensor 107 in the return tube 142. Sensors that are located within and/or monitored by the ECGE system 103 are referred to herein as “ECGE sensors.” Sensors 107 that are located within and/or monitored by the ventilator 101 are referred to herein as “ventilator sensors.”

The ECGE system 103 may also include a heat exchanger, connectors, switches and/or a bladder reservoir. In some embodiments, the heat exchanger is a water heater. In some embodiments, the heat exchanger is integrated with the membrane lung 146. For example, a water heater heat exchanger may pump heated water into the membrane lung 146. In some embodiments, the bladder receives blood passively drained by gravity from the patient 150 via the access tube 140. The bladder may be collapsible and may act as complaint reservoir. In some embodiments, the pump 144 draws blood from the bladder. In some embodiments, a switch provides the pump 144 access to the blood in the bladder. For example, in some embodiments, the bladder and pump 144 are connected by a trip-switch mechanism so that if the pump flow exceeds venous drainage, the balder collapses to inhibit pump flow.

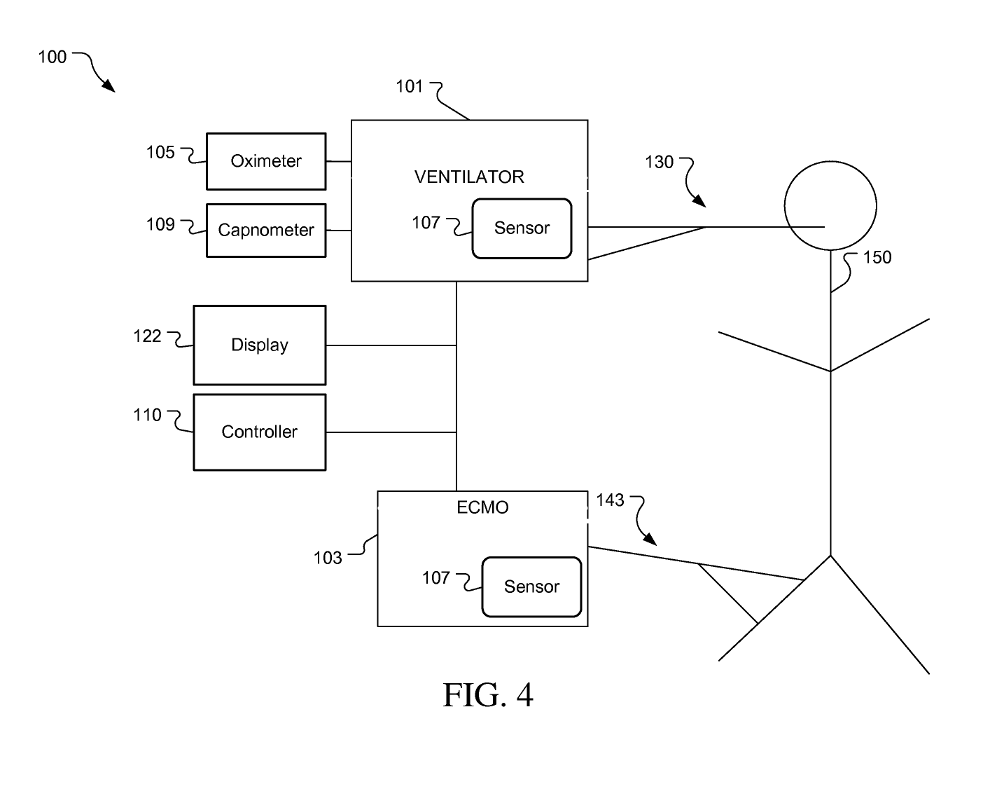

An oximeter 105 is connected to a patient oximeter sensor 107. In some embodiments, the oximeter 105 is a completely separate and an independent component from the ventilator 101 and the ECGE system 103 as illustrated in FIG. 4. In an alternative embodiment, as illustrated in FIG. 1, the oximeter 105 is part of the ventilator-ECGE system 100. For example, as illustrated in FIG. 1, the oximeter 105 is part of the pneumatic system 102.

In some embodiments, the ventilator-ECGE system 100 includes an oximeter 105. The oximeter 105, if utilized, determines an oxygen gas saturation level of blood in the patient 150 based on the patient readings taken by the pulse oximeter sensor 107 during treatment of a patient 150 by the ventilator-ECGE system 100. The oximeter 105 sends the measured oxygen saturation level of the blood of the patient 150 to a controller 110. In some embodiments, the controller 110 determines and/or monitors generated sensor output from the oximeter sensor 107 and the ventilator-ECGE system 100, such as a SpO₂ of the patient 150. As discussed above the sensor output may be taken directly from sensor data or derived from sensor data. In some embodiments, controller 110 sends the data to the display 122 for displaying the generated sensor output from the oximeter sensor 107.

A capnometer 109, if utilized, is in data communication with ventilator-ECGE system 100. This communication allows the ventilator-ECGE system 100 and capnometer 109 to send data, instructions, and/or commands to each other. In some embodiments, the capnometer 109 is in communication with controller 110 and/or the processor 116 of the ventilator-ECGE system 100. In some embodiments, as illustrated in FIG. 1, the capnometer 109 is a component of the ventilator-ECGE system 100. In other embodiments, the capnometer 109 is a separate component from the ventilator-ECGE system 100 and in data communication with the ventilator-ECGE system 100, as illustrated in FIG. 4. In an example, as illustrated in FIG. 1, the capnometer 109 is part of the pneumatic system 102.

Capnometer 109 monitors the concentrations of carbon dioxide in the respiratory gas with a carbon dioxide sensor 107. In some embodiments, the carbon dioxide sensor 107 is located in the ventilator breathing circuit 130. The carbon dioxide sensor 107 allows the capnometer 109 to monitor in real-time or quasi-real-time the concentration of CO₂ in the gas transiting the carbon dioxide sensor 107. Using this in conjunction with flow and/or volume signals, the capnometer 109 and/or the ventilator-ECGE system 100 may calculate volumetric carbon dioxide (VCO₂), end-tidal carbon dioxide (E_{T}CO₂), and minute volume. The VCO₂ may measure the amount of CO₂ removed from the blood by the ECGE device and/or may measure the amount of CO₂ exhaled by patient through the patient circuit of the ventilator. In one embodiment, capnometer 109 generates a capnogram or capnograph with received data and/or sensor output. In some embodiments, the controller 110 determines and/or monitors generated sensor output from the carbon dioxide sensor 107 and the ventilator-ECGE system 100.

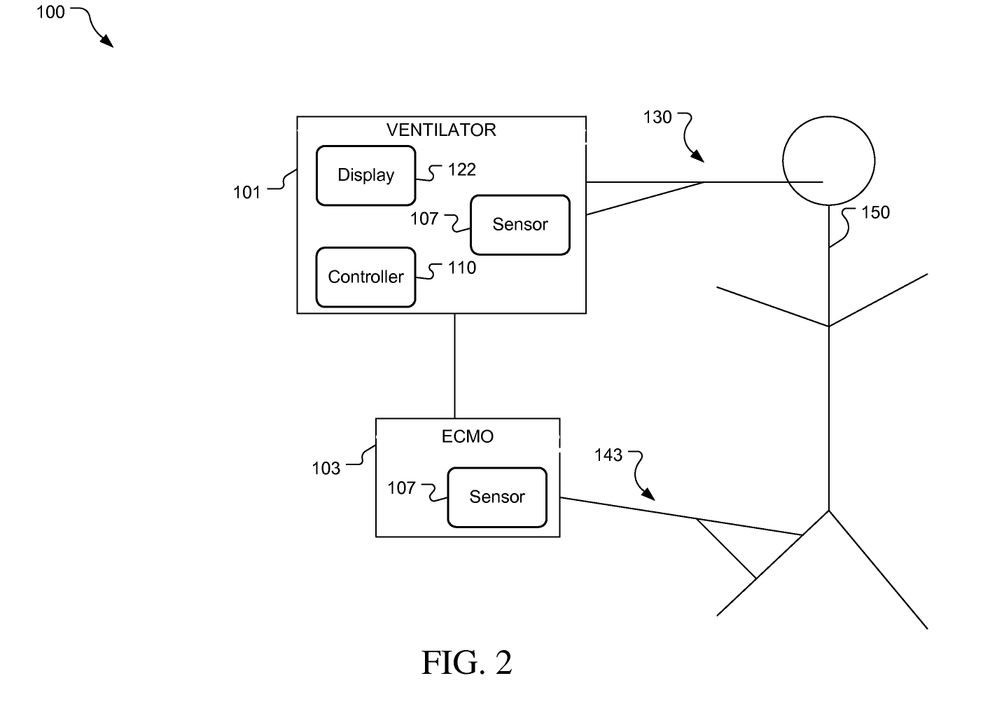

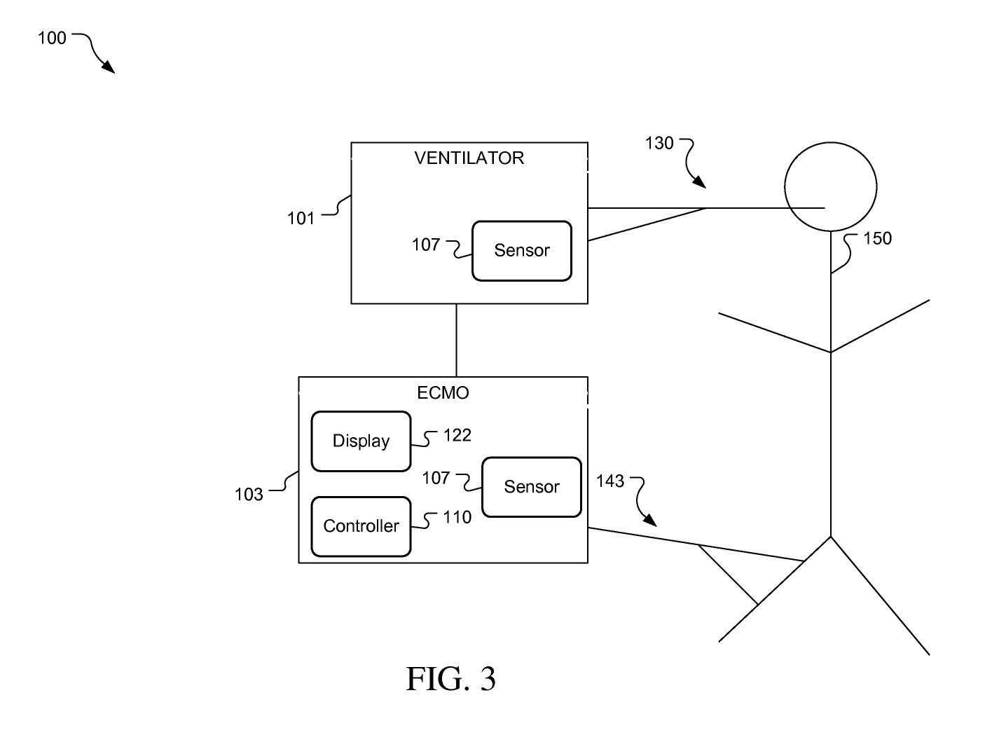

The ventilator-ECGE system 100 includes one or more controllers 110. In some embodiments, the controller 110 is part of the ventilator 101 as illustrated in FIGS. 1 and 2. In some embodiments, the controller 110 is part of the ECGE system 103 as illustrated in FIG. 3. In other embodiments, the controller 110 is separate and independent of both the ventilator 101 and the ECGE system 103 as illustrated in FIG. 4. In other embodiments, the ventilator 101 and the ECGE system 103 each have their own controller 110. In these embodiments, the controllers 110 communicate between each other. For example, the two controllers 110 may be wirelessly connected or connected via a cable and may send and/or receive data, instructions, and/or commands.

The controller 110 is operatively coupled with pneumatic system 102, signal measurement and acquisition systems (including sensors 107), membrane lung 146, pump 144, and/or an operator interface 120 that may enable an operator to interact with the ventilator 101 (e.g., change ventilator settings, select operational modes, view monitored parameters, etc.). In some embodiments, the controller 110 controls the pneumatic system 102, signal measurement and acquisition systems (including sensors 107), membrane lung 146, pump 144, and/or operator interface 120. In some embodiments, the controller 110 sends sensor output to the display module 122. In further embodiments, the controller 110 processes the sensor output to show trends, create charts, create graphs, group related data, and/or even create picotograms for display by the display module 122 to convey information to the operator in a more useful, faster to decipher, and/or easier to comprehend manner.

In some embodiments, the controller 110 communicates with and/or controls several different subsystems that may be distributed and networked (wired or any type of wireless) and will operate autonomously to provide their designated outcomes while in continuous communication with the integrated controller 110 to send and/or receive data and/or settings or supervisory control commands. The candidate subsystems include: data acquisition and transmission capabilities, ventilators 101, lung dialysis machines 103, oxygen and CO₂ monitoring (e.g., oximeter 105 and capnography 109), hemodynamic monitoring devices to monitor the patient's parameters (such as measurement of systemic, pulmonary arterial and venous pressures, and of cardiac output). The controller 110 will obtain necessary sensor output and clinical data, and uses designated supervisory control algorithms to determine and issue settings and/or commands to the applicable device(s).

In one embodiment, the operator interface 120 of the ventilator-ECGE system 100 includes one or more display modules 122 communicatively coupled to the ventilator-ECGE system 100. In some embodiments, the display module 122 is part of the ventilator 101 as illustrated in FIGS. 1 and 2. In some embodiments, the display module 122 is part of the ECGE system 103 as illustrated in FIG. 3. In other embodiments, the display module 122 is separate and independent of both the ventilator 101 and the ECGE system 103 as illustrated in FIG. 4. In other embodiments, the ventilator 101 and the ECGE system 103 each have their own display module 122.

Display module 122 may provide various input screens, for receiving clinician input, and various display screens, for presenting useful information to the clinician. In one embodiment, the display module 122 is configured to include a graphical user interface (GUI). The GUI may be an interactive display, e.g., a touch-sensitive screen or otherwise, and may provide various windows and elements for receiving input and interface command operations. Alternatively, other suitable means of communication with the ventilator 101 and/or the ventilator-ECGE system 100 may be provided, for instance by a wheel, keyboard, mouse, or other suitable interactive device. Thus, operator interface 120 may accept commands and input through display module 122 and/or or another communication device. The controller 110 may receive the operator input from the display modules 122.

Display module 122 may also provide useful information in the form of various ventilatory data, ECGE data, capnometer data, and/or oximeter data. The useful information may be derived from data collected by a processor, such as processor 116, and the useful information may be displayed to the clinician in the form of graphs, wave representations, lists, check lists, pie graphs, text, or other suitable forms of graphic display. For example, patient data may be displayed on the GUI and/or display module 122. In some embodiments, the display module 122 issues at least one of the appropriate message and the appropriate recommendation entry.

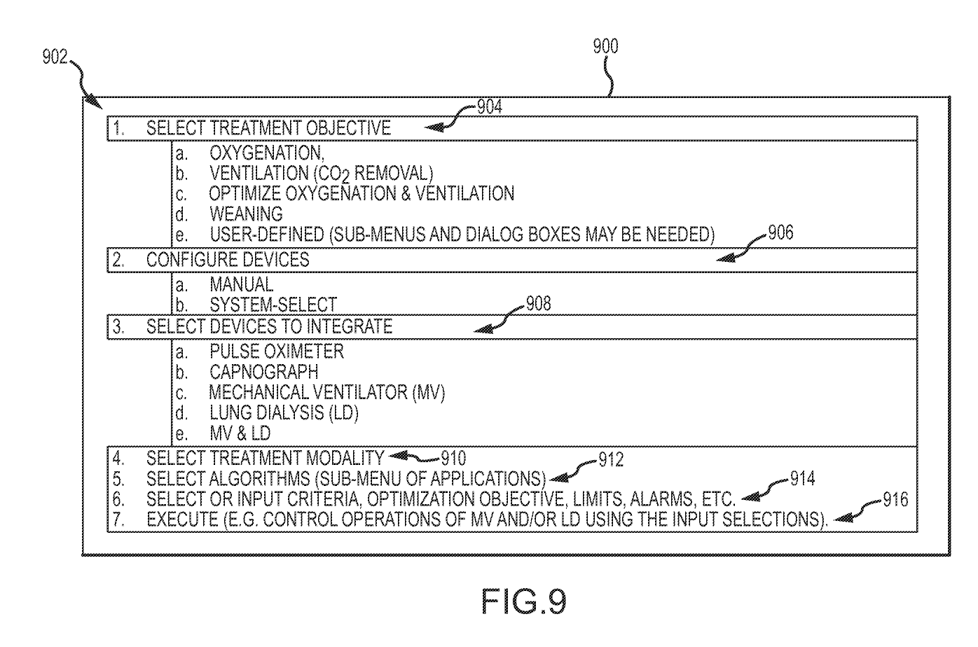

In some embodiments, the user interface is menu driven and proceeds from the desired treatment objective in a top down algorithmic fashion. In these embodiments, the menu could take the user through processes in a sequential manner and provide the next menu based on the choices made in the previous steps. For example, FIG. 9 illustrates an embodiment of a graphical user interface 900 displaying a selectable drop down menu 902. In some embodiments, the selectable drop down menu 902 is a in a top down algorithmic menu. In some embodiments, the menu 902 includes treatment objectives 904, configuration options 906, device selection options 908, treatment modality options 910, algorithm options 912, threshold options 914, and/or execution options 916.

In some embodiments, the display module 122 may be a remote display module. Additionally or alternatively, useful information and/or ventilator parameters may be communicated to and displayed on an additional remote display module and/or on a remote monitoring system coupled via any suitable means to the ventilator-ECGE system 100, such as a tablet or PC. The remote display module or remote monitoring system are not physically attached to the ventilator-ECGE system 100 and may be in the same room or over a mile away from the patient 150 or the ventilator-ECGE system 100. In some embodiments, the display module 122 and/or remote monitoring display system displays a sweep gas flow rate, PₐCO₂, pH, CO₂ removal rate, FᵢO₂ of sweep gas, ventilator FiO₂, blood flow, cardiac output, PₐO₂, OI index, PₐO₂/FᵢO₂, HCO₃⁻, SaO₂ minute ventilation, peak inspiratory pressure, tidal volume, respiratory rate, PEEP, SpO₂, E_{T}CO₂, VCO₂, alarms, recommendations, and/or notifications.

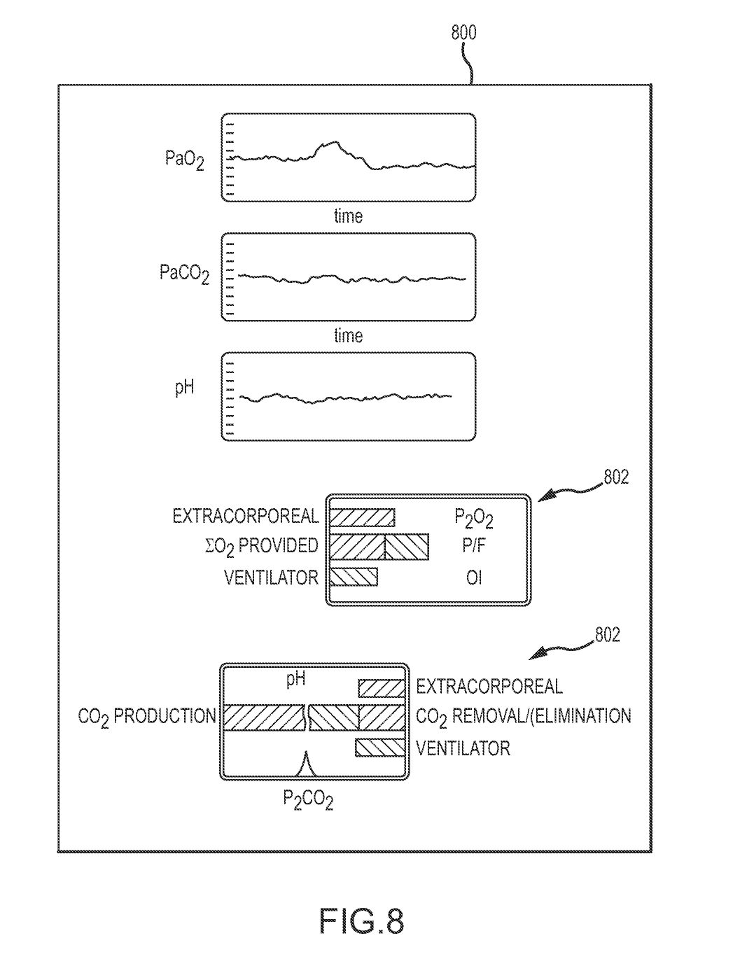

The display module 122 receives and displays data from both the ventilator 101 and the ECGE system 103. In some embodiments, as discussed above, the displayed data is received from the controller 110 and/or processor 116. Taken together, the data monitored collectively by the ventilator 101 and by the ECGE system 103 provide an extensive “picture” of the respiratory-mechanical and the gas-exchange properties (health) of the respiratory-organ system of the patient 150. In some embodiments, displayed information is formatted by one or more information control algorithms. In some embodiments, a simple alpha character display will suffice; in other instances a two-dimensional pictogram may be more informative; and in other instances a three-dimensional pictogram may be the better choice. Further, as discussed above trends to indicate the interaction between the lung dialysis machine and the mechanical ventilation as they relate to oxygenation and ventilation as determined by the controller 110 and/or processor 116 may be displayed by the display module 122. In some embodiments, related data from the ventilator 101 and ECGE system 103 is grouped together to convey information to the operator in a more useful, faster to decipher, and/or easier to comprehend manner, as illustrated in FIG. 8. FIG. 8 illustrates an embodiment of a graphical user interface 800 displaying grouped related data 802 from the ventilator 101 and the ECGE system 103.

In further embodiments, as discussed above, the display module 122 displays a menu driven user interface. For example, FIG. 9 illustrates an embodiment of a graphical user interface 900 displaying a selectable drop down menu 902. In some embodiments, the display module 122 displays a menu including treatment objectives 904, configuration options 906, device selection options 908, treatment modality options 910, algorithm options 912, threshold options 914, and/or execution options 916. The treatment objectives 904 may include oxygenation, ventilation, optimize oxygenation and ventilation, weaning, and/or user defined objectives. The configuration options 906 may include manual and system-select options. The device selection options 908 may include pulse oximeter, capnometer (also referred to as a capnograph), ventilator, ECGE system, and/or ventilator and ECGE system. These lists are exemplary only and are not meant to be limiting. Any suitable options for controlling and/or managing the ventilation and oxygenation of the patient with the ventilator-ECGE system 100 may be displayed and/or provided by the menu.

Controller 110 includes memory 112, one or more processors 116, storage 114, and/or other components of the type commonly found in command and control computing devices. In some embodiments, controller 110 also includes a compare module 124, a manage module 126, and/or a notification module 118 as illustrated in FIG. 1. In alternative embodiments, a manage module 126, compare module 124, and/or a notification module 118 may be located in other components of the ventilator 101, such as the pressure generating system 102 (also known as the pneumatic system 102). In other embodiments, the manage module 126, compare module 124, and/or a notification module 118 may be located in the ECGE system 103. In yet other embodiments, the manage module 126, compare module 124, and/or a notification module 118 may be located in a controller 110 separate and independent of the ECGE system 103 and the ventilator 101.

The memory 112 includes non-transitory, computer-readable storage media that stores software that is executed by the processor 116 and which controls the operation of the ventilator-ECGE system 100. In an embodiment, the memory 112 includes one or more solid-state storage devices such as flash memory chips. In an alternative embodiment, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

In some embodiments, the ventilator-ECGE system 100 includes a compare module 124. The compare module 124 compares sensor output to at least one threshold. In some embodiments, the compare module 124 determines or monitors trends in sensor output and compares these trends to at least one threshold. In some embodiments, the compare module 124 compares sensor output from ventilator sensors 107 to one or more thresholds. In other embodiments, the compare module 124 compares sensor output from ECGE sensors 107 to one or more thresholds. In further embodiments, the compare module 124 compares sensor output from a capnometer sensor 107 and/or an oximeter sensor 107 to one or more thresholds. In other embodiments, the compare module 124 compares sensor output from ventilator sensors 107 and ECGE sensors 107 to the same threshold. In further embodiments, the compare module 124 compares sensor output from one or more ventilator sensors 107 to a first threshold and sensor output from ECGE sensors 107 to a second threshold. In additional embodiments, the compare module 124 compares sensor output from one or more ventilator sensors 107 to a first plurality of thresholds and compares sensor output from the ECGE sensors 107 to a second plurality of thresholds. The first plurality of thresholds may be equivalent, overlap, or be entirely different from the second plurality of thresholds. Based on this comparison, the compare module 124 determines if the generated sensor output breached the at least one threshold.

In some embodiments, the at least one threshold is received from operator input. In some embodiments, the operator input is received from the ventilator 101. In other embodiments, the operator input is received from the ECGE system 103. In some embodiments, the operator input is received from a graphical user interface. In some embodiments, the at least one threshold is determined by the ventilator-ECGE system 100. In some embodiments, the ventilator-ECGE system 100 determines the one or more threshold based on predetermined settings and patient characteristics, such as patient age, patient height, patient weight, patient gender, patient disease state, ventilation mode, and/or sweep gas flow rate. This list is exemplary only and is not meant to be limiting.

In some embodiments, the one or more threshold is an end tidal carbon dioxide (E_{T}CO₂) level, a volumetric carbon dioxide (VCO₂) level, a blood oxygen saturation (SpO₂) level, a partial pressure of carbon dioxide in arterial blood (PₐCO₂) level, a pH level, a blood flow level, a partial pressure of oxygen in the arterial blood (PₐO₂) level, a ratio of the partial pressure of oxygen in the arterial blood divided by a fraction of inspired oxygen level (PₐO₂/FᵢO₂), and/or an oxygenation index (OI). The oxygenation index is the fraction of inspired oxygen multiplied by the mean airway pressure, the product of which is divided by the partial pressure of oxygen in arterial blood as shown below:

$$OI = \frac{{F}_{i} {O}_{2} * {M}_{PAW}}{Pa {O}_{2}} .$$

In other embodiments, the one or more threshold is a combination of two or more of the following parameters/levels: an end tidal carbon dioxide (E_{T}CO₂) level; a volumetric carbon dioxide (VCO₂) level or rate; a blood oxygen saturation (SpO₂) level; a partial pressure of carbon dioxide in arterial blood (PₐCO₂) level; a pH level; a blood flow level; a partial pressure of oxygen in the arterial blood (PₐO₂) level; a ratio of the partial pressure of oxygen in the arterial blood divided by a fraction of inspired oxygen level (PₐO₂/FᵢO₂); and/or an oxygenation index (OI).

For example, in some embodiments, a threshold may include a PₐCO₂ level outside of 36 mm of Hg to 46 mm of Hg and/or a blood pH outside of 7.3 to 7.5. In other examples, the threshold may include a SaO₂ outside of an 85-95% range, a PₐO₂ outside of a 45-55 mmHg range, and/or a Hematocrit below 5% of a normal baseline. These thresholds are exemplary only and are not meant to be limiting. Any suitable threshold for oxygenating and/or ventilating a patient may be utilized by the ventilator-ECGE system 100.

In some embodiments, the ventilator-ECGE system 100 includes a manage module 126. The manage module 126 manages the treatment of the patient. The treatment of the patient as used herein refers to the oxygenation and/or ventilation of the patient. The manage module 126 manages the treatment of the patient 150 by adjusting, if needed, one or more ventilator settings and/or one or more ECGE settings. The manage module 126 manages the oxygenation of the patient 150 by adjusting one or more ventilator settings and/or one or more ECGE settings, if needed, based on information received from the compare module 124. In alternative embodiments, the manage module 126 determines one or more necessary adjustments based on information received from the compare module 124 and sends this information to the display module 122 for display. In alternative embodiments, the manage module 126 determines one or more appropriate adjustments based on information from the compare module 124 and sends or communicates the appropriate adjustment to the notification module 118. In some embodiments, the manage module 126 detects improper treatment of a patient based on sensor output from a ventilator sensor 107 and/or from an ECGE sensor 107. The improper treatment is improper oxygenation and improper ventilation of a patient 150. In additional embodiments, the manage module 126 determines the sensor output that implicated the improper treatment and communicates the sensor output that implicated the improper treatment to the notification module.

For example, in some embodiments, if the manage module 126 determines that the PₐCO₂, pH, and/or CO₂ removal rate need to be adjusted based on information from the compare module 124, the manage module 126 adjusts the sweep gas flow rate to achieve the desired PₐCO₂, pH, and/or CO₂ removal rate. In another example, in some embodiments, if the manage module 126 determines that the PₐO₂ and/or blood flow rate need to be adjusted based on information from the compare module 124, the manage module 126 adjusts the FᵢO₂ of the sweep gas and/or a blood flow rate of the pump 144 if a pump is utilized in the ECGE system 103 to achieve the desired PₐO₂ and/or blood flow rate. In an additional example, in some embodiments, if the manage module 126 determines that the E_{T}CO₂ and/or VCO₂ need to be adjusted based on information from the compare module 124, the manage module 126 adjusts the minute ventilation, peak inspiration pressure, tidal volume, and/or respiratory rate to achieve the desired E_{T}CO₂ and/or VCO₂.

In another example, in some embodiments, if the manage module 126 determines that the SpO₂ needs to be adjusted based on information from the compare module 124, the manage module 126 adjusts the FᵢO₂ delivered to the patient's lungs, minute volume, peak inspiratory pressure, the PEEP, FᵢO₂ concentration in the sweep gas, sweep gas flow rate, and/or blood flow rate to achieve the desired SpO₂. However, under some conditions the deficiency in blood oxygenation and/or ventilation (such as CO₂ removal) is caused by physiologic etiologies that adjustments of ventilator parameters may not provide timely and/or efficient improvements. If the manage module 126 determines that the SpO₂ needs to be adjusted based on information from compare module 124 after a change in one or more of above listed ventilator parameters has been made, the manage module 126 may choose to increase the above listed ECGE parameters instead of or in combination with the above listed ventilator parameters. In some embodiments, the following parameters or variables are monitored to determine proper oxygenation PaCO₂, pH, E_{T}CO₂, and/or VCO₂. In some embodiments, the following parameters or variables are monitored to determine proper ventilation blood flow, PₐO₂, and/or SpO₂. These lists are exemplary and are not limiting. Any suitable parameter for monitoring the oxygenation and/or ventilation of the patient 150 may be monitored by the ventilator-ECGE system 100.

The manage module 126 may have different set goals or multiple goals in managing the oxygenation of the patient 150. For example, the manage module 126 may be set to optimize ventilation, to optimize oxygenation, and/or to protect the lungs by reducing pressure applied to lungs. Further, the manage module 126 may be able to achieve the same goal by adjusting different parameters or a combination of different parameters. This flexibility allows the ventilator-ECGE system 100 to adjust the settings of ventilator 101 and/or ECGE system 103 if more or less CO₂ removal is desirable or if more or less oxygenation is desirable based on patient needs. As discussed above a patient with ARDS may only be able to tolerate a low amount of pressure ventilation. Further, as discussed above, if the ECGE system 103 has been utilized for an extended period of time, the manage module 126 can choose to reduce the blood flow rate in the ECGE system 103 to prevent necrosis or ischemia.

For example, if manage module 126 determines that one or more setting needs to be adjusted to optimize CO₂ removal, the manage module 126 can increase the sweep gas flow rate and/or increase minute ventilation (by either increasing the respiratory rate and/or the tidal volume). In another example, if manage module 126 determines that one or more setting needs to be adjusted to protect the lungs, the manage module 126 can decrease tidal volume or peak inspiratory pressure for an increase in sweep flow rate. However, in this example, if the increase in sweep flow rate does not result in the desired CO₂ removal, an increase in minute ventilation can provide for additional CO₂ removal. In another example, if the manage module 126 determines that one or more settings need to be adjusted to optimize oxygenation, the manage module 126 can increase blood flow rate, FᵢO₂ concentration in the sweep gas, PEEP, and/or FᵢO₂ delivered to the lungs of the patient 150 to optimize the oxygenation of the patient 150. Additionally, the manage module 126 may suggest or make changes to reduce the reliance of the patient 150 on the ECGE system 103 or the ventilator 101.

In some embodiments, the manage module 126 utilizes algorithms to develop an adaptive control scheme. In some embodiments, the manage module 126 utilizes adaptive algorithms with online reinforced learning paradigms to develop adaptive control schemes. For example, the manage module 126 may make automatic adaptive adjustments to ventilator settings to optimize ventilation and/or oxygenation and may when a saturation effect is detected (no observable change caused by ventilator adjustments over a predetermined period of time) or when a declining trend in efficiency or efficacy of ventilator parameter adjustments is detected based on information from the compare module 124.

In some embodiments where one of the ventilator 101 and the ECGE system 103 are not being actively utilized, the manage module 126 will determine based on information from the compare module 124 that the inactive system (the ventilator 101 or the ECGE system 103) should be activated to treat the patient 150 in addition to the other treatment. In other embodiments, where both the ventilator 101 and the ECGE system 103 are being utilized to treat the patient 150, the manage module 126 will determine based on information from the compare module 124 that one of the active systems (the ventilator 101 or the ECGE system 103) should be deactivated or shut down and should not be utilized to treat the patient 150. In some embodiments, the manage module 126 after making a determination will activate or deactivate the appropriate system. In alternative embodiments, the manage module 126 after making a determinations will send a recommendation to activate or deactivate the appropriate system to the notification module 118.

The settings of the ventilator 101 can be adjusted to either load or unload cardiac output dependent upon the phasing of positive pressure and the phase of the heart beat (systolic or diastolic). For example, if positive pressure is applied by the ventilator 101 at the point when blood is flowing out of the heart, cardiac output can be increased (or the imposed load on the heart can be decreased). Conversely, if positive pressure is applied by the ventilator 101 when the heart is filling with blood, an additional load is imposed on the heart and cardiac output will decrease.

Accordingly, in some embodiments, the ventilator-ECGE system 100 includes a synchronize module 128. The synchronize module 128 monitors or detects the timing of cardiac systolic and diastolic cycles and synchronizes the triggering of inspiration and exhalation of the patient 150 with the ventilator 101 based on the detected timing to modify a patient's cardiac load. In some embodiments, the synchronize module 128 further adjusts the detected timing of the cardiac systolic and diastolic cycles to account for any appropriate delay caused by measurement, signal transfer, and/or derivations. In some embodiments, the adjustment is based on physiological and biomechanical models and signal measurement and data acquisition characteristics of the system used.

In some embodiments, the synchronize module 128 utilizes an ECG to determine or monitor the timing of cardiac systolic and diastolic cycles of the patient's heart. In some embodiments, the synchronize module 128 monitors a patient heartbeat or heart rate to determine or monitor the timing of cardiac systolic and diastolic cycles of the patient's heart. As used herein the terms “pulse rate,” “heart rate,” “heart beat,” and “pulse” are considered to be interchangeable in the present disclosure and in the claims. While “pulse rate,” “heart rate,” “heartbeat,” and “pulse” refer to different measurements, it is understood by a person of skill in the art that any of these measurements may be used for the purposes of this disclosure and for the purposes of the claims. The synchronize module 128 may utilize any suitable system or method for determining the heartbeat of the patient 150. The synchronize module 128 synchronizes the triggering of inspiration and exhalation of the patient 150 with the ventilator 101 based on the detected timing to reduce a patient's cardiac load by directing the ventilator 101 to trigger an inspiration when blood is flowing out of the heart and/or to trigger an exhalation when blood is flowing into the heart. The synchronize module 128 synchronizes this triggering based on the detected timing of the cardiac systolic and diastolic cycles. In some embodiments, cardio respiratory synchronization may be scheduled using an adaptive phase detection and phase delay algorithm that utilizes streaming ECG waveforms (detecting the timing of cardiac systolic/diastolic cycles) and appropriate delay times to synchronize breath delivery phasing (inspiration triggering and expiration cycling) to optimize a desired cardio respiratory objective. The synchronization is not intended to change the ventilator's breath triggering and cycling timing in such a way that may jeopardize the respiratory needs of the patient. It will be an optimization process to determine phase adjustments within physiologically acceptable temporal limits.

In further embodiments, the process may be optimized by providing a menu of ventilator parameters such as minute volume, PEEP, target rate, oxygen mix, I:E ratio, inspiratory duration, expiratory duration, and/or etc., that may be used individually or in combinations to make adaptive ventilation adjustments to achieve better synchrony. In yet other embodiments, for passive patients, the respiratory rate for ventilator 101 may be derived as a proper integer ratio (fixed or user settable) of the patient's heart rate (Respiratory Rate=integer (heart rate/selected ratio)). In additional embodiments, for actively breathing patients, a form of synchronized intermittent Mandatory ventilation (SIMV) breath delivery may be used with allowable ventilator adjustments to optimize synchrony.

As discussed above, arteriovenous ECGE systems utilize the patient's heart to pump the patient's blood through the blood circuit 143 of the ECGE system 103. Accordingly, the arteriovenous ECGE system increases the length and the resistance through which the heart has to pump the blood volume. This extended length and increased resistance increases the cardiac load on the patient's heart. Therefore, previously, patients that were unable to sustain additional cardiac load (e.g., patients at risk for cardiac failure) were excluded from the use of arteriovenous ECGE systems. Accordingly, in some embodiments, the ventilator-ECGE system 100 may utilize the synchronize module 128 to offset at least a portion, if not all, of the increased cardiac load created by the arteriovenous ECGE system. Accordingly, ventilator-ECGE system 100 can now be applied to patients that were previously excluded from using an arteriovenous ECGE system based on the ventilator 101 offsetting of the increased cardiac load. However, the synchronize module 128 of the ventilator-ECGE system 100 can be utilized to decrease the cardiac load of any patient 150 being treated with the ventilator-ECGE system 100.

The ventilator-ECGE system 100 may further include a notification module 118. As may be appreciated, multiple ventilator parameters and ECGE system parameters may be monitored and evaluated in order to maintain the oxygenation and/or ventilation of the patient 150. In addition, when an adjustment is implicated to maintain the oxygenation of the patient 150, many clinicians may not be aware of adjustments that could be made to maintain the oxygenation and/or ventilation of the patient 150. As such, upon determination that an adjustment is necessary (such as communication with manage module 126) to maintain oxygenation and/or ventilation of the patient 150, the notification module 118 may be configured to notify the clinician that improper oxygenation and/or ventilation is detected, that an adjustment is necessary, and/or to provide suggestions of appropriate adjustments to the clinician or operator for managing the oxygenation and/or ventilation of the patient 150. For example, notification module 118 may be configured to notify the clinician by displaying a notification on the display module 122 and/or within a window of a graphical user interface (GUI). In additional embodiments, the notification is communicated to and/or displayed on a remote monitoring system communicatively coupled to ventilator-ECGE system 100. In some embodiments, the notification is any audio and/or visual notification. Alternatively, in an automated embodiment, the notification module 118 communicates with a controller 110 so that the recommendation may be automatically implemented to manage oxygenation and/or ventilation of the patient 150.

In order to accomplish the various aspects of the notification message display, the notification module 118 may communicate with various other components and/or modules. For instance, notification module 118 may be in communication with controller 110, compare module 124, manage module 126, synchronize module 128, one or more sensors 107, oximeter 105, capnometer 109 and/or any other suitable module or component of the ventilator-ECGE system 100. That is, notification module 118 may receive an indication that an improper oxygenation and/or ventilation is detected and/or that an adjustment is needed to maintain oxygenation and/or ventilation of the patient 150 by any suitable means. In addition, notification module 118 may receive information regarding one or more parameters or sensor output that implicate the improper oxygenation and/or ventilation and/or the need for the adjustment and information regarding the ventilator settings and/or parameters, ECGE settings and/or parameters, patient characteristics, and/or patient treatment from the controller 110, compare module 124, manage module 126, synchronize module 128, one or more sensors 107, oximeter 105, capnometer 109 and/or any other suitable module or component of the ventilator-ECGE system 100. Further, according to some embodiments, the notification module 118 may have access to a patient's diagnostic information (e.g., regarding whether the patient has ARDS, COPD, asthma, emphysema, or any other disease, disorder, or condition). In some embodiments the notification module 118 determines an appropriate message based on the improper treatment and determines an appropriate recommendation entry based on the improper treatment. Further, in some embodiments, the notification module 118 issues at least one of the appropriate message and the appropriate recommendation entry. In additional embodiments, the notification module 118 determines the appropriate message based at least in part on the sensor output that implicated the improper treatment.

Notification module 118 may provide a notification according to a hierarchical structure such that a notification is presented as a message and/or a recommendation entry and may be initially presented in summarized form and, upon clinician selection, an additional detailed message and/or recommendation entry is displayed. According to alternative embodiments, a notification is initially presented and, upon clinician selection, a recommendation entry is displayed. Alternatively or additionally, the notification may be simultaneously displayed with the recommendation entry in any suitable format or configuration.

Specifically, according to embodiments, the notification alerts the clinician as to the detection of a patient condition, a change in patient condition, or an effectiveness of ventilator and/or ECGE treatment. For example, the notification may alert the clinician that improper oxygenation and/or ventilation was detected, that an adjustment is necessary to maintain oxygenation and/or ventilation, and/or the sensor output or threshold that was breached that necessitated the needed adjustment. The notification may further alert the clinician regarding the particular parameter(s) that implicated improper oxygenation and/or ventilation (e.g., PₐCO₂, pH, blood flow, PₐO₂, SpO₂, E_{T}CO₂, VCO₂, and/or etc.)

Additionally, according to embodiments, the recommendation entry provides various suggestions to the clinician for managing the oxygenation and/or ventilation of the patient 150. That is, if an adjustment is needed, the recommendation entry may suggest that the clinician consider adjusting a sweep gas flow rate, FᵢO2 of the sweep gas, blood flow, minute ventilation, peak inspiratory pressure, tidal volume, respiratory rate, PEEP, FᵢO₂ delivered to the patient's lungs and/or etc. According to additional embodiments, the recommendation entry is based on a particular breached parameter(s) (e.g., PₐCO₂, pH, blood flow, PₐO₂, E_{T}CO₂, VCO₂, SpO₂, and/or etc.) that implicated a needed adjustment. Additionally or alternatively, the recommendation entry may be based on current ventilatory settings (e.g., breath type) such that suggestions are directed to a particular patient's treatment. Additionally or alternatively, the recommendation entry may be based on a diagnosis and/or other patient attributes. Further still, the recommendation entry may include a primary recommendation entry and a secondary recommendation entry.

Notification module 118 generates a notification via any suitable means. For example, the notification message may be provided as a tab, banner, dialog box, or other similar type of display. Further, the notification messages may be provided along a border of the graphical user interface, near an alarm display or bar, or in any other suitable location. A shape and size of the notification message may further be optimized for easy viewing with minimal interference to other displays. The notification message may be further configured with a combination of icons and text such that the clinician may readily identify the message as a notification message.

Notification module 118, in some embodiments, generates one or more recommendation entry via any suitable means. The one or more recommendation entry or messages may provide suggestions and information regarding addressing a detected condition and may be accessible from the notification message. For example, the one or more recommendation entry may identify the parameters that implicated improper ventilation and/or oxygenation, may provide suggestions for adjusting one or more parameters to manage oxygenation and/or ventilation of the patient 150, may provide suggestions for checking equipment or patient position, or may provide other helpful information. Specifically, the one or more recommendation entries may provide suggestions and information regarding patient oxygenation.

According to embodiments, based on the particular parameters that were breached, the notification module 118 provides suggestions for managing oxygenation. In some embodiments, the notification module 118 provides suggestions for maintaining oxygenation based on the breached parameter. That is, if adjustment is necessary, the one or more recommendation entries may include suggestions or recommendations for the following:
increase the sweep flow rate based on at least one of the following sensor outputs: PₐCO₂, pH, E_{T}CO₂, and VCO₂;
decrease the sweep flow rate based on at least one of the following sensor outputs: PₐCO₂, pH, E_{T}CO₂, and VCO₂;
increase the FiO₂ of the sweep gas based on at least one of the following sensor outputs: PₐO₂, blood flow, and SpO₂;
decrease the FiO₂ of the sweep gas based on at least one of the following sensor outputs: PₐO₂, blood flow, and SpO₂;
increase the blood flow through an ECGE system based on at least one of the following sensor outputs: PₐO₂, blood flow, and SpO₂;
decrease the blood flow through the ECGE system based on at least one of the following sensor outputs: PₐO₂, blood flow, and SpO₂;
increase the FiO₂ based on at least one of the following sensor outputs: PₐO₂, blood flow, and SpO₂;
decrease the FiO₂ based on at least one of the following sensor outputs: PₐO₂, blood flow, and SpO₂;
increase the PEEP based on at least one of the following sensor outputs: PₐO₂, blood flow, and SpO₂;
decrease the PEEP based on at least one of the following sensor outputs: PₐO₂, blood flow, and SpO₂;
increase the peak inspiration pressure based on at least one of the following sensor outputs: PₐCO₂, pH, E_{T}CO₂, and VCO₂;
decrease the peak inspiration pressure based on at least one of the following sensor outputs: PₐCO₂, pH, E_{T}CO₂, and VCO₂;
increase the tidal volume based on at least one of the following sensor outputs: PₐCO₂, pH, E_{T}CO₂, and VCO₂;
decrease the tidal volume based on at least one of the following sensor outputs: PₐCO₂, pH, E_{T}CO₂, and VCO₂;
increase the respiratory rate based on at least one of the following sensor outputs: PₐCO₂, pH, E_{T}CO₂, and VCO₂;
decrease the respiratory rate based on at least one of the following sensor outputs: PₐCO₂, pH, E_{T}CO₂, and VCO₂;
increase the minute ventilation based on at least one of the following sensor outputs: PₐCO₂, pH, E_{T}CO₂, and VCO₂; and
decrease the minute ventilation based on at least one of the following sensor outputs: PₐCO₂, pH, E_{T}CO₂, and VCO₂.

According to still other embodiments, the recommendation message includes a primary entry or message and a secondary entry or message. That is, a primary message may provide notification of the condition detected and/or suggestions that are specifically targeted to the detected condition based on the particular parameters that implicated a needed adjustment. Alternatively, the primary message may provide suggestions that may provide a higher likelihood of mitigating the improper oxygenation and/or ventilation. The secondary message may provide more general suggestions and/or information that may aid the clinician in further addressing and/or mitigating the improper oxygenation and/or ventilation. For example, the primary message may provide a specific suggestion for adjusting a particular parameter to mitigate a breached parameter (e.g., consider increasing FᵢO₂). Alternatively, the secondary message may provide general suggestions for addressing the breached parameter.

Additionally or alternatively, the one or more recommendation entries may also be based on a secondary condition or current ventilator settings for the patient 150 and/or the type of asynchrony detected. For example, if SpO₂ breached a threshold even after a change to a ventilator setting had been made, then the one or more recommendation entries may suggest that the clinician make specific changes to the ECGE settings, such as increase the FᵢO₂ of the sweep flow gas.

The detection of a breached parameter as described above informs the ventilator-ECGE system 100 that the patient 150 is not receiving the proper oxygenation and/or ventilation. Accordingly, in some embodiments, this information is utilized in a message and/or recommendation entry to the clinician. In an alternative embodiment, the ventilator-ECGE system 100 automatically adjusts a parameter based on the detected breached parameter.

Table 1 below lists various examples of primary and secondary recommendations for various breached parameters that may change due to secondary conditions, such as patient disease state.

TABLE 1
Entry messages based on breached parameter and/or secondary conditions.

| Breached | Secondary | | |
|---|---|---|---|
| Parameter | Condition | Primary Entry | Secondary Entry |
| Low SpO₂ | Patient does not | Consider increasing minute | Consider increasing |
| | meet criteria for | ventilation, peak inspiratory | sweep gas flow rate |
| | ARDS, ALI, or | pressure, tidal volume, PEEP, | |
| | VILI | FᵢO₂, and/or respiratory rate | |
| Low SpO₂ | Patient suffers | Consider increasing sweep gas | Consider increasing |
| | from severe lung | flow rate | minute ventilation, peak |
| | injury: ARDS, | | inspiratory pressure, |
| | ALI, or VILI | | tidal volume, PEEP, |
| | | | FᵢO₂, and/or respiratory |
| | | | rate |
| Low | Hyperventilation | Consider adjusting minute | Check integrity of gas |
| E_{T}CO₂ | (high respiratory | ventilation, PEEP, FᵢO₂, and/or | collection |
| | rate) | respiratory rate | device/process; |
| | | Check integrity of ECGE | Consider adjusting |
| | | membrane; Consider increasing | sweep gas flow rate |
| | | sweep gas flow rate | |
| High | Hypoventilation | Consider increasing minute | Check integrity of |
| E_{T}CO₂ | | ventilation, PEEP, FᵢO₂, and/or | ECGE membrane; |
| | | respiratory rate | Consider increasing |
| | | | sweep gas flow rate |
| High | Hyperventilation | Consider decreasing minute | Check integrity of gas |
| VCO₂ | | ventilation, adjusting PEEP, | collection |
| | | and/or respiratory rate. | device/process; |
| | | Check integrity of ECGE | Consider decreasing |
| | | membrane | sweep gas flow rate |
| Low | Hypoventilation | Consider increasing minute | Check integrity of |
| VCO₂ | | ventilation, PEEP, FᵢO₂, and/or | ECGE membrane; |
| | | respiratory rate | Consider increasing |
| | | | sweep gas flow rate |
| High | Patient has been | Check integrity of ECGE | Consider increasing |
| PaCO₂ | treated with | membrane; Consider increasing | minute ventilation, |
| | ECGE system for | sweep gas flow rate | PEEP, FᵢO₂, and/or |
| | an extended | | respiratory rate |
| | period of time | | |
| High | Hypoventilation | Consider increasing minute | Check integrity of |
| PaCO₂ | | ventilation, PEEP, FᵢO₂, and/or | ECGE membrane; |
| | | respiratory rate | Consider increasing |
| | | | sweep gas flow rate |
| High | Heart Failure | Consider adjusting minute | Consider adjusting |
| PaCO₂ | | ventilation, PEEP, FᵢO₂, and/or | ECGE settings, |
| | | respiratory rate | Consider adjusting |
| | | | sweep gas flow rate; |
| | | | Consider checking O₂ |
| | | | consumption |
| High | Low cardiac | Consider adjusting ECGE settings, | Consider adjusting |
| PaCO₂ | output | Consider adjusting sweep gas flow | minute ventilation, peak |
| | | rate; | inspiratory pressure, |
| | | | tidal volume, PEEP, |
| | | | FᵢO₂, and/or respiratory |
| | | | rate |
| ECGE | Pulmonary | Consider checking oxygenator, | Consider adjusting |
| Blood | hypertension | membrane/inlet/outlet port | minute ventilation, peak |
| Flow | | | inspiratory pressure, |
| outside of | | | tidal volume, PEEP, |
| expected | | | FᵢO₂, and/or respiratory |
| range | | | rate |
| ECGE | Renal | Consider checking cannula and | none |
| Blood | complications | tubing (wrong size, bleeding, | |
| Flow | | malposition, clotting, | |
| outside of | | decannulation, etc.) | |
| expected | | | |
| range | | | |
| High | Hypercapnea | Consider increasing sweep gas | Consider increasing |
| PaCO₂ | (adult patient) | flow rate; Check integrity of | minute ventilation, peak |
| | | ECGE membrane; | inspiratory pressure, |
| | | | tidal volume, PEEP, |
| | | | FᵢO₂, and/or respiratory |
| | | | rate |
| High | Impaired | Consider adjusting ECGE settings; | Consider adjusting |
| PaCO₂ | cardiovascular | Consider checking for air | minute ventilation, peak |
| | function | embolism; ECGE inlet | inspiratory pressure, |
| | | obstruction; ECGE gas-blood leak; | tidal volume, PEEP, |
| | | high FiO₂ | FᵢO₂, and/or respiratory |
| | | | rate |
| Low | Hypercapnea | Consider increasing sweep gas | Consider increasing |
| pH | (adult patient) | flow rate; Check integrity of | minute ventilation, peak |
| | | ECGE membrane; | inspiratory pressure, |
| | | | tidal volume, PEEP, |
| | | | FᵢO₂, and/or respiratory |
| | | | rate |
| Low | Metabolic | Consider increasing sweep gas | Consider increasing |
| pH | acidosis | flow rate; Check integrity of | minute ventilation, peak |
| | (refractory) | ECGE membrane; | inspiratory pressure, |
| | | | tidal volume, PEEP, |
| | | | FᵢO₂, and/or respiratory |
| | | | rate |
| Low SₚO₂ | Peak airway | Consider increasing ventilator | Consider increasing |
| or Low | pressures below | FiO₂ and/or PEEP | blood flow rate and/or |
| PₐO₂ | VILI criterion and | | sweep flow FiO₂ |
| | FiO₂ less than | | |
| | 70% | | |
| Low SₚO₂ | Patient diagnosed | Consider increasing blood flow | Consider increasing |
| or Low | with ARDS, ALI, | rate and/or sweep flow FiO₂ | ventilator FiO₂ and |
| PₐO₂ | or peak airway | | decreasing tidal volume |
| | pressures above | | |
| | VILI criterion | | |
| Low SₚO₂ | Arteriovenous | Consider increasing sweep flow | Consider increasing |
| or Low | system | FiO₂ | ventilator FiO₂ and/or |
| PₐO₂ | | | PEEP |
| High | Peak airway | Consider increasing minute | Consider increasing |
| E_{T}CO₂ | pressures below | ventilation (inspiratory pressure, | sweep flow rate |
| | VILI criterion | tidal volume, and respiratory rate) | |
| High | Patient diagnosed | Consider increasing sweep flow | Consider increasing |
| E_{T}CO₂ | with ARDS, ALI, | rate | respiratory rate |
| | or peak airway | | |
| | pressures above | | |
| | VILI criterion | | |
| Low | Minute | Consider decreasing minute | Consider decreasing |
| E_{T}CO₂ | ventilation > 50 | ventilation (inspiratory pressure, | sweep gas flow rate |
| | ml/kg | tidal volume, and respiratory rate) | |
| Low | Minute | Consider decreasing sweep gas | Consider decreasing |
| E_{T}CO₂ | ventilation < 50 | flow rate | minute ventilation |
| | ml/kg | | (inspiratory pressure, |
| | | | tidal volume, and |
| | | | respiratory rate) |
| High | Peak airway | Consider increasing minute | Consider increasing |
| PaCO₂ | pressures below | ventilation (inspiratory pressure, | sweep flow rate |
| | VILI criterion | tidal volume, and respiratory rate) | |
| High | Patient diagnosed | Consider increasing sweep flow | Consider increasing |
| PaCO₂ | with ARDS, ALI, | rate | respiratory rate |
| | or Ppeak airway | | |
| | pressures above | | |
| | VILI criterion | | |
| High | PaCO₂ low | Consider decreasing sweep flow | Consider decreasing |
| pH | | rate | minute ventilation |
| | | | (inspiratory pressure, |
| | | | tidal volume, and |
| | | | respiratory rate) |
| Low | Peak airway | Consider increasing minute | Consider increasing |
| pH | pressures below | ventilation (inspiratory pressure, | sweep flow rate |
| | VILI criterion | tidal volume, and respiratory rate) | |
| Low | Patient diagnosed | Consider increasing sweep flow | Consider increasing |
| pH | with ARDS, ALI, | rate | minute ventilation |
| | or peak airway | | (inspiratory pressure, |
| | pressures above | | tidal volume, and |
| | VILI criterion | | respiratory rate) |

In some embodiments, the notification message is associated with a primary and secondary message and with one or more recommendation entries that may be associated with the secondary message. That is, a primary message may provide an alert that the patient 150 is not being properly oxygenated and/or ventilated and may further provide one or more potential causes, such as a breached parameter. Alternatively, an alert may be separately provided, indicating that that the patient 150 is not being properly oxygenated and/or ventilated, and the primary message may provide the one or more potential causes. According to additional or alternative embodiments, the secondary message provides the one or more recommendation entries and/or information that may aid the clinician in further addressing and/or mitigating the improper oxygenation and/or ventilation. For example, the secondary message may recommend addressing the improper oxygenation and/or ventilation by adjusting a ventilator parameter, by adjusting an ECGE parameter, by activating or deactivating the ventilator 101, by activating or deactivating the ECGE system 103, and/or etc. Notification module 118 may also be configured such that notification (including alerts, primary messages, and/or secondary messages) may be displayed in a partially transparent window or format. The transparency may allow for notifications and/or entries to be displayed such that normal ventilator GUI and respiratory data may be visualized behind the messages. This feature may be particularly useful for displaying detailed messages. Notification module 118 may also be configured such that notification (including alerts, primary messages, and/or secondary messages) may be displayed on or within a pictorial representation of the lung. The lung representation may be two-dimensional, three-dimensional, and/or animated. As described previously, notifications may be displayed in areas of the display screen that are either blank or that cause minimal distraction from the respiratory data and other graphical representations provided by the GUI. However, upon selective expansion of a notification, such as an entry or message, respiratory data, ECGE data, and graphs may be at least partially obscured. As a result, translucent display may provide the detailed notification such that it is partially transparent. Thus, graphical and other data may be visible behind the detailed alarm message.

Additionally, notifications may provide immediate access to the display and/or settings screens associated with the detected condition. For example, an associated parameter settings screen may be accessed from a notification, such as a message or recommendation entry, via a hyperlink such that the clinician may address the detected condition as necessary. An associated parameter display screen may also be accessed such that the clinician may view clinical data associated with the detected condition in the form of charts, graphs, or otherwise. That is, according to embodiments, the clinician accesses the ventilator, ECGE, capnometer, and/or oximeter data that implicated the detected condition for verification purposes. For example, when a parameter has been breached, depending on the particular ventilatory parameters that were breached, the clinician may be able to access ventilatory settings for addressing the needed adjustment (e.g., a settings screen for adjusting respiration rate, PEEP, blood flow, etc.) and/or to view associated parameters that also implicated a needed adjustment (e.g., a graphics screen displaying historical flow waveforms, current tidal volume, and/or waveforms illustrating the breached parameter).

According to embodiments, upon viewing the notification and/or recommendation messages, upon addressing a breached parameter by adjusting one or more settings or otherwise, or upon manual selection, the notification is cleared from the graphical user interface. According to some embodiments, notification module 118 clears the one or more notifications or messages from the graphical user interface if a setting is changed on the ventilator-ECGE system 100, such as sweep gas flow. In further embodiments, notification module 118 clears the one or more messages from the graphical user interface if a setting change was performed by the operator and a threshold was not breached for a predetermined amount of time or number of breaths. In further embodiments, notification module 118 clears the one or more messages from the graphical user interface if the threshold breach does not occur again during a predetermined amount of time or breaths. In some embodiments, the notification module 118 clears the one or more messages from the graphical user interface upon user selection.

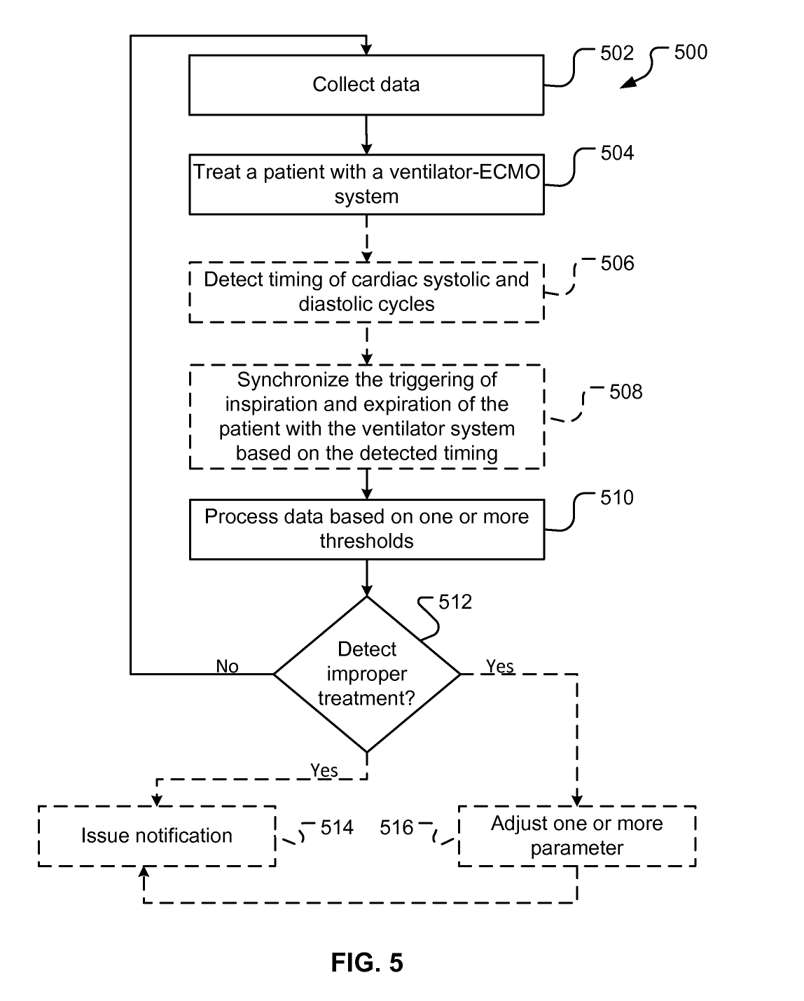

FIG. 5 is a flow chart illustrating an embodiment of a method 500 for managing the treatment of a patient with a medical system. The medical system of method 500 may be any medical system for treating a patient that includes a ventilator and an ECGE system, such as a ventilator-ECGE system. While having different meanings, the “ventilator-ECGE system” and the “medical system” may be referred to during method 1100 interchangeably.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

The illustrated embodiment of the method 500 depicts a method for detecting improper oxygenation and/or ventilation of a patient connected to a ventilator-ECGE system. Method 500 begins with collecting data operation 502. The ventilator-ECGE system during collecting data operation 502 receives data regarding one or more ventilator settings and/or one or more ECGE settings from operator input. For example, the ventilator-ECGE system may be configured to provide ventilation to a patient and/or oxygenation of a patient. As such, the input received may include predicted or ideal body weight (PBW or IBW), trigger sensitivity, PEEP, one or more thresholds, sweep gas flow rate, blood flow, and/or etc. The ventilator-ECGE system during collecting data operation 502 also receives sensor output and/or data from the monitoring of one or more sensors. The sensors may be ventilator sensors, ECGE sensors, capnometer sensors, oximeter sensors, and/or any other suitable sensors for treatment of a patient with a ventilator-ECGE system. In some embodiments, the ventilator-ECGE system during collecting data operation 502 also receives derived data from a processor based on sensor output. A parameter refers to any factor, characteristic, sensor output, derived output, and/or measurement associated with the treatment of a patient by the ventilator-ECGE system, whether monitored by the ventilator-ECGE system or input by the operator/clinician. As used herein the terms “parameter” and “setting” may be utilized interchangeably in the disclosure and claims. In some embodiments, the collected data is transmitted by sensors. In some embodiments, data or sensor output regarding flow rate, circuit pressure, flow pattern, inspiratory time setting (T_{I}), sweep gas flow rate, blood flow, pH, PₐCO₂, E_{T}CO₂, VCO₂, and/or etc., may be collected from the sensors, operator interface, controller, and/or processor.

At treatment operation 504, the ventilator-ECGE system provides ventilation and/or oxygenation to a patient. In some embodiments, the ventilator-ECGE system during treatment operation 504 delivers breathing gas to a patient with a ventilator system based on one or more ventilator settings. In some embodiments, the ventilator-ECGE system during treatment operation 504 removes carbon dioxide from a patient's bloodstream and adds a gas mixture to the patient's bloodstream to oxygenate the blood stream of the patient with the ECGE system based on one or more ECGE settings. Accordingly, the ventilator-ECGE system may provide just ventilation with the ventilator, just oxygenation with the ECGE system, or a combination of overlapping or simultaneous ventilation and oxygenation by the ventilator with the ECGE system. In some embodiments, the ventilator-ECGE system provides ventilation and/or oxygenation to a patient based on operator input. In other embodiments, the ventilator-ECGE system provides ventilation and/or oxygenation to a patient based on data from the collecting data operation 502. In some embodiments, the ventilator-ECGE system includes an oximeter and/or capnometer.

During treatment operation 504, the ventilator-ECGE system communicates information between the ventilator system and the ECGE system. In some embodiments, during treatment operation 504, the ventilator-ECGE system communicates information between the ventilator system, the ECGE system, an oximeter, and/or a capnometer. The communication may flow in one direction or in both directions. Further, the communication may include data, signals, commands, and/or instructions. Further, the communication between the systems may be wireless or wired.

While the patient is being treated with the ventilator-ECGE system, the ventilator-ECGE system may conduct various data processing operations. For example, at processing operation 510, the ventilator-ECGE system collects and/or derives various parameters data from sensor output. In some embodiments, the processing operation 510 is performed by the manage module 126, compare module 124, controller 110, processor 116 and/or pneumatic system 102 that are discussed in detail above. For example, as described above, the ventilator may collect data regarding parameters including PₐCO₂, pH, blood flow, PₐO₂, E_{T}CO₂, VCO₂, SpO₂, and/or etc. Additionally, the ventilator may derive various parameter data based on the collected data. For examples, parameters such as IBW-predicted T_{I}, volume, respiratory resistance, respiratory compliance, detected patient triggers, detected patient cycles, OI, and/or etc. may be derived from other collected data by the ventilator during processing operation 510. Generated sensor output and/or derived output may be trended continuously for a patient. Additionally, the ventilator-ECGE system may generate various graphical representations of the collected and/or derived parameter data, e.g., flow waveforms, pressure waveforms, pressure-volume loops, flow-volume loops, pictorial representation of the lung, and/or etc.

The ventilator-ECGE system during processing operation 510 compares the sensor output or the derived sensor output to one or more thresholds. In some embodiments, the ventilator-ECGE system during processing operation 510 determines or monitors trends in sensor output and compares these trends to at least one threshold.

In some embodiments, the ventilator-ECGE system during processing operation 510 compares sensor output from ventilator sensors to one or more thresholds. In other embodiments, ventilator-ECGE system during processing operation 510 compares sensor output from ECGE sensors to one or more thresholds. In further embodiments, the ventilator-ECGE system during processing operation 510 compares sensor output from a capnometer sensor and/or an oximeter sensor to one or more thresholds. In other embodiments, the ventilator-ECGE system during processing operation 510 compares sensor output from ventilator sensors and ECGE sensors to the same one or more thresholds. In further embodiments, the ventilator-ECGE system during processing operation 510 compares sensor output from one or more ventilator sensors to a first threshold and sensor output from ECGE sensors to a second threshold. In additional embodiments, the ventilator-ECGE system during processing operation 510 compares sensor output from one or more ventilator sensors to a first plurality of thresholds and compares sensor output from the ECGE sensors to a second plurality of thresholds. The first plurality of thresholds may be equivalent, overlap, or be entirely different from the second plurality of thresholds.

In some embodiments, the one or more threshold is an E_{T}CO₂ level, a VCO₂ level, a SpO₂ level, a PₐCO₂ level, a pH level, a blood flow level, a PₐO₂ level, a PₐO₂/FᵢO₂, tidal volume, blood flow level, peak inspiratory pressure, and/or an OI. In other embodiments, the one or more threshold is a combination of two or more of the following parameters/levels: an E_{T}CO₂ level; a VCO₂ level; a SpO₂ level; a PₐCO₂ level; a pH level; a blood flow level; a PₐO₂ level; blood flow level; a PₐO₂/FᵢO₂; tidal volume; peak inspiratory pressure; and/or an OI.

According to some embodiments, at detect improper treatment operation 512 the ventilator-ECGE system determines whether improper oxygenation and/or ventilation is implicated based on the comparison of the sensor data to one or more thresholds performed during the processing operation 510. In some embodiments, at detect improper treatment operation 512 the ventilator-ECGE systems determine whether improper treatment is implicated based on whether the sensor output or parameters derived from the sensor output breached one or more thresholds. In some embodiments, the detect improper treatment operation 512 is performed by the manage module 126, controller 110, processor 116 and/or pneumatic system 102 that are discussed in detail above.

In some embodiments, at detect improper treatment operation 512 the ventilator-ECGE system may determine whether improper treatment is implicated based on a predetermined frequency of occurrence of a breached parameter. For example, in one embodiment, the ventilator-ECGE system at detect improper treatment operation 512 determines that improper ventilation and/or oxygenation is implicated when a parameter is breached for two or more consecutive breaths or for a predetermined time period, such as 10 milliseconds or more.

If improper ventilation and oxygenation are not implicated, the detect improper treatment operation 512 may return to collecting data operation 502. However, in some embodiments, the ventilator-ECGE system continuously performs the collecting data operation 502, the treatment operation 504, processing operation 510, and detect improper treatment operation 512. If improper ventilation and/or oxygenation is implicated, detect improper treatment operation 512 may proceed to either issuing notification operation 514 or adjusting operation 516. In some embodiments, the ventilator-ECGE system is configured to perform either or both the issuing notification operation 514 and the adjusting operation 516. In some embodiments, the adjusting operation 516 is performed only upon user selection. In alternative embodiments, the ventilator-ECGE system is configured to perform the issuing notification operation 514 and not the adjusting operation 516.

The thresholds listed above are just one example list of possible conditions that could be used to indicate improper ventilator and/or oxygenation in the detect improper treatment operation 512. Any suitable list of conditions for determining the occurrence of improper ventilation and/or oxygenation may be utilized by the detect improper treatment operation 512. As may be appreciated, the ventilator-ECGE system may determine whether improper ventilation and/or oxygenation is implicated at detect improper treatment operation 512 via any suitable means. Indeed, any of the above described parameters may be evaluated according to various thresholds for detecting improper ventilation and/or oxygenation. Further, the disclosure regarding specific parameters as they may implicate improper treatment is not intended to be limiting. In fact, any suitable parameter or sensor output may be monitored and evaluated for detecting improper oxygenation and/or ventilation within the spirit of the present disclosure. As such, if improper oxygenation and/or ventilation is implicated via any suitable means, the detect improper treatment operation 512 may proceed to the issuing notification operation 514 and/or the adjusting operation 516.

At issuing notification operation 514, the ventilator-ECGE may alert the clinician via any suitable means that improper treatment (i.e., improper ventilation and/or oxygenation) has been implicated. For example, according to embodiments, the ventilator may display a notification including a message and/or a recommendation entry regarding the detection and/or cause of improper ventilation and/or oxygenation on the GUI. According to alternative embodiments, the ventilator may communicate the notification, including the message and/or the recommendation entry, to a remote monitoring system communicatively coupled to the ventilator-ECGE system. According to alternative embodiments, the issued notification is any visual and/or audio notification.

According to embodiments, the notification alerts the clinician that improper ventilation and/or oxygenation has been detected and, optionally, provides information regarding any parameter(s) that implicated the improper ventilation and/or oxygenation. According to additional embodiments, the notification may provide one or more suggestions for mitigating improper oxygenation and/or ventilation. In some embodiments, the suggestions or entries are based on information from processing operation 510. According to further embodiments, the one or more suggestions may be based on the patient's particular settings (e.g. breath type, flow pattern, flow rate, PEEP, and/or etc.) and/or diagnosis. According to some embodiments, the clinician may access one or more parameter settings and/or display screens from the notification via a hyperlink or otherwise for addressing improper ventilation and/or oxygenation. According to additional or alternative embodiments, a clinician may remotely access one or more parameters and/or display screens from the notification via a hyperlink or otherwise for remotely addressing improper ventilation and/or oxygenation.

In some embodiments, method 500 includes an adjusting operation 516. At adjusting operation 516, the ventilator-ECGE system adjusts one or more ventilator settings and/or one or more ECGE settings. At adjusting operation 516, the ventilator-ECGE system adjusts one or more ventilator settings and/or one or more ECGE settings based on comparison of the sensor output to one or more thresholds by the processing operation 510. In some embodiments, the adjusting operation 516 is performed by the manage module 126, controller 110, processor 116 and/or pneumatic system 102 that are discussed in detail above.

For example, in some embodiments, if the ventilator-ECGE system during adjusting operation 516 determines that the PₐCO₂, pH, and/or CO₂ removal rate need to be adjusted based on information from the comparison made by the processing operation 510, the ventilator-ECGE system adjusts the sweep gas flow rate to achieve the desired PₐCO₂, pH, and/or CO₂ removal rate. In another example, in some embodiments, if the ventilator-ECGE system during adjusting operation 516 determines that the PₐO₂ and/or SpO₂ need to be adjusted based on information from the comparison made by the processing operation 510, the ventilator-ECGE system adjusts the FᵢO₂ of the sweep gas and/or a blood flow rate of the pump 144 if a pump is utilized in the ECGE system to achieve the desired PₐO₂ and/or blood flow rate. In an additional example, in some embodiments, if the ventilator-ECGE system during adjusting operation 516 determines that the E_{T}CO₂ and/or VCO₂ need to be adjusted based on information from the comparison made by the processing operation 510, the ventilator-ECGE system adjusts the minute ventilation, peak inspiration pressure, tidal volume, and/or respiratory rate to achieve the desired E_{T}CO₂ and/or VCO₂.

In another example, in some embodiments, if the ventilator-ECGE system during adjusting operation 516 determines that the SpO₂ needs to be adjusted based on information from the comparison made by the processing operation 510, the ventilator-ECGE system adjusts the FᵢO₂ delivered to the patient's lungs, the PEEP, FᵢO₂ concentration in the sweep gas, and/or blood flow rate to achieve the desired SpO₂. However, under some conditions the deficiency in blood oxygenation and/or ventilation (such as CO₂ removal) is caused by physiologic etiologies that adjustments of ventilator parameters may not provide timely and/or efficient improvements. If the ventilator-ECGE system during adjusting operation 516 determines that the SpO₂ needs to be adjusted based on information from the comparison made by the processing operation 510 after a change in one or more of above listed ventilator parameters has been made, the ventilator-ECGE system may choose to increase the above listed ECGE parameters instead of or in combination with the above listed ventilator parameters.

The ventilator-ECGE system during adjusting operation 516 may have different set goals or multiple goals in managing the oxygenation and/or ventilation of the patient. For example, the ventilator-ECGE system during adjusting operation 516 may be set to optimize ventilation, to optimize oxygenation, and/or to protect the lungs by reducing pressure applied to lungs. Further, the ventilator-ECGE system during adjusting operation 516 may be able to achieve the same goal by adjusting different parameters or a combination of different parameters. This flexibility allows the ventilator-ECGE system during adjusting operation 516 to adjust the settings of the ventilator and/or the ECGE system if more or less ventilation is desirable or if more or less oxygenation is desirable based on patient needs. As discussed above, a patient with ALI may only be able to tolerate a low amount of pressure ventilation. Further, if the ECGE system has been utilized for an extended period of time, the ventilator-ECGE system during adjusting operation 516 may choose to reduce the blood flow rate in the ECGE system to prevent or reduce the likelihood of necrosis.

For example, if the ventilator-ECGE system during adjusting operation 516 determines that one or more setting needs to be adjusted to optimize ventilation, the ventilator-ECGE system can increase the sweep flow rate and/or increase minute ventilation (by either increasing the respiratory rate and/or the tidal volume). In another example, if the ventilator-ECGE system during adjusting operation 516 determines that one or more settings need to be adjusted to protect the lungs, the ventilator-ECGE system can decrease tidal volume or peak inspiratory pressure for an increase in sweep flow rate. However, in this example, if the increase in sweep flow rate does not result in the desired CO₂ removal, an increase in minute ventilation can provide for additional CO₂ removal. In another example, if the ventilator-ECGE system during adjusting operation 516 determines that one or more setting needs to be adjusted to optimize oxygenation, the ventilator-ECGE system can increase blood flow rate, FᵢO₂ concentration in the sweep gas, PEEP, and/or FᵢO₂ delivered to the lungs of the patient to optimize the oxygenation of the patient's blood.

In some embodiments, the ventilator-ECGE system during adjusting operation 516 utilizes algorithms to develop an adaptive control scheme. In some embodiments, the ventilator-ECGE system during adjusting operation 516 utilizes adaptive algorithms with online reinforced learning paradigms to develop adaptive control schemes. For example, the ventilator-ECGE system during adjusting operation 516, may make automatic adaptive adjustments to ventilator settings to optimize ventilation and/or oxygenation and may when a saturation effect is detected (no observable change caused by ventilator adjustments over a predetermined period of time) or when a declining trend in efficiency or efficacy of ventilator parameter adjustments is detected based on information from the processing data operation 510, make an ECGE setting adjustment.

In some embodiments, where one of the ventilator and the ECGE system are not being actively utilized, the ventilator-ECGE system during adjusting operation 516 will activate the inactive system (the ventilator or the ECGE system) to treat the patient. In other embodiments, where both the ventilator and the ECGE system are being utilized to treat the patient, the ventilator-ECGE system during adjusting operation 516 will deactivate or shut down systems (the ventilator or the ECGE system). In some embodiments, the ventilator-ECGE system during adjusting operation 516 will activate or deactivate the appropriate system.

In some embodiments, after performing the adjusting operation 516, the ventilator-ECGE system will perform the issuing notification operation 514. According to embodiments, the ventilator-ECGE system during adjusting a issuing notification operation 514, issues a notification to alert the clinician that improper ventilation and/or oxygenation was detected and mitigated with an appropriate adjustment, and optionally, may provide information regarding any parameter(s) that implicated the improper ventilation and/or oxygenation and the implemented adjustment. In some embodiments, the issuing notification operation 514 is performed by the notification module 118, display module 122, controller 110, processor 116 and/or pneumatic system 102 that are discussed in detail above. In some embodiments, the method 500 further includes a timing operation 506 and a synchronizing operation 508. In some embodiments, the timing operation 506 and the synchronizing operation 508 are performed by the synchronize module 128, controller 110, processor 116 and/or pneumatic system 102 that are discussed in detail above. As discussed above, the settings of the ventilator can be adjusted to either load or unload cardiac output dependent upon the phasing of positive pressure and the phase of the heart beat (systolic or diastolic). Accordingly, the ventilator-ECGE system during the timing operation 506 monitors or detects the timing of cardiac systolic and diastolic cycles. In some embodiments, the ventilator-ECGE system during the timing operation 506 monitors an ECG to detect the timing of cardiac systolic and diastolic cycles of the patient's heart. In other embodiments, the ventilator-ECGE system during the timing operation 506 monitors a patient heartbeat or heart rate to detect the timing of cardiac systolic and diastolic cycles of the patient's heart. The ventilator-ECGE system during the timing operation 506 may utilize any suitable system or method for determining the heartbeat of the patient.

In some embodiments, the ventilator-ECGE system during the timing operation 506 adjusts the detected timing of the cardiac systolic and diastolic cycles to account for any appropriate delay caused by measurement, signal transfer, and/or derivations. In some embodiments, the adjustment is based on physiological and biomechanical models and signal measurements and data acquisition characteristics of the system used.

The ventilator-ECGE system during the synchronizing operation 508 synchronizes the triggering of inspiration and/or exhalation of the patient with the ventilator based on the detected timing to reduce a patient's cardiac load. The ventilator-ECGE system during the synchronizing operation 508 synchronizes the triggering of inspiration and exhalation of the patient with the ventilator based on the detected timing to reduce a patient's cardiac load by directing the ventilator to trigger an inspiration when blood is flowing out of the heart and to trigger an exhalation when blood is flowing into the heart. The ventilator-ECGE system during the synchronizing operation 508 synchronizes this triggering based on the detected timing of the cardiac systolic and diastolic cycles. In some embodiments, cardio respiratory synchronization may be scheduled using an adaptive phase detection and phase delay algorithm that utilizes streaming ECG waveforms (detecting the timing of cardiac systolic/diastolic cycles) and appropriate delay times to synchronize breath delivery phasing (inspiration triggering and expiration cycling) to optimize a desired cardio respiratory objective. The synchronization is not intended to change the ventilator's breath triggering and cycling timing in such a way that may jeopardize the respiratory needs of the patient. It will be an optimization process to determine phase adjustments within physiologically acceptable temporal limits.

In further embodiments, the ventilator-ECGE system during the synchronizing operation 508 may optimize synchronization by providing a menu of ventilator parameters such as minute volume, PEEP, target rate, oxygen mix, I:E ratio, inspiratory duration, expiratory duration, and/or etc., that may be used individually or in combinations to make adaptive ventilation adjustments to achieve better synchrony. In yet other embodiments, for passive patients, the respiratory rate for the ventilator may be derived as a proper integer ratio (fixed or user settable) of the patient's heart rate (Respiratory Rate=integer (heart rate/selected ratio) by the ventilator-ECGE system during the synchronizing operation 508. In additional embodiments, for actively breathing patients, a form of synchronized intermittent Mandatory ventilation (SIMV) breath delivery may be used with allowable ventilator adjustments to optimize synchrony by the ventilator-ECGE system during the synchronizing operation 508.

As discussed above, arteriovenous ECGE systems utilize the patient's heart to pump the patient's blood through the blood circuit of the ECGE system. Accordingly, the arteriovenous ECGE system increases the length and resistance through which the heart has to pump the blood volume. This extended length and increased resistance increases the cardiac load on the patient's heart. Therefore, previously, patients that are unable to sustain additional cardiac load (e.g., patients at risk for cardiac failure) were excluded from the use of arteriovenous ECGE systems. Accordingly, in some embodiments, the ventilator-ECGE system utilizes the timing operation 506 and the synchronizing operation 508 to offset at least a portion, if not all, of the increased cardiac load created by the arteriovenous ECGE system. Accordingly, ventilator-ECGE system 100 can now be applied to patients that that were previously excluded from using an arteriovenous ECGE system based on the ventilator offsetting of the increased cardiac load. However, the timing operation 506 and the synchronizing operation 508 of method 500 can be utilized by the ventilator-ECGE system to decrease the cardiac load of any patient being treated with the ventilator-ECGE system.

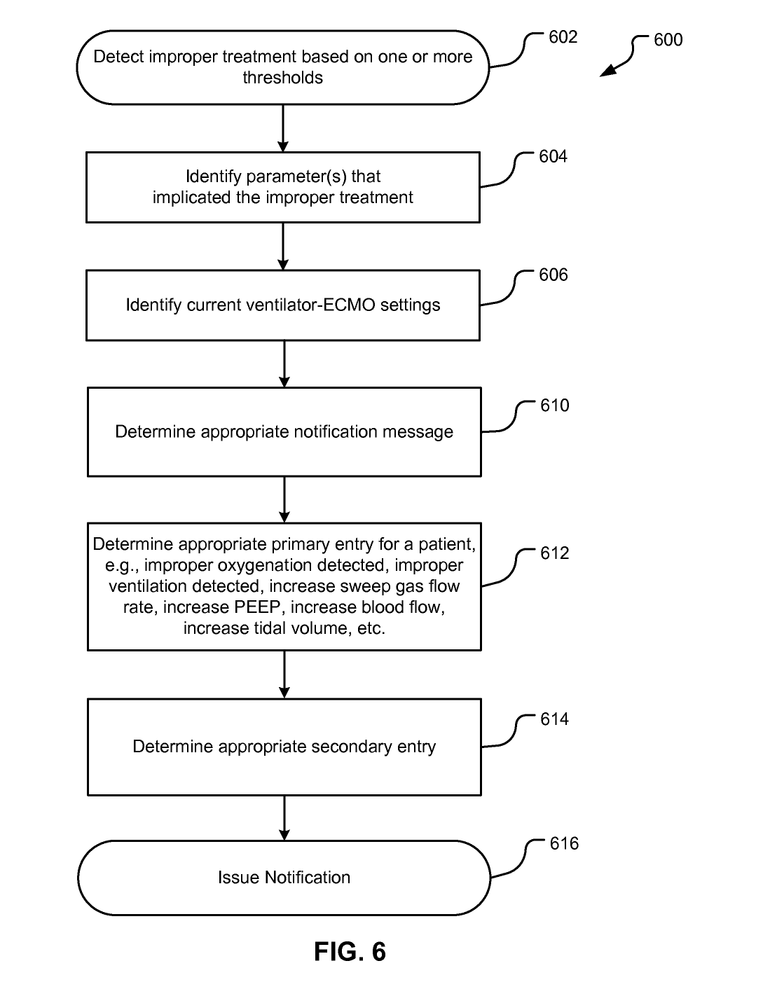

FIG. 6 illustrates an embodiment of a method 600 for issuing a notification upon detecting an implication of improper ventilation and/or oxygenation.

As should be appreciated, the particular steps and methods described herein are not exclusive and, as will be understood by those skilled in the art, the particular ordering of steps as described herein is not intended to limit the method, e.g., steps may be performed in differing order, additional steps may be performed, and disclosed steps may be excluded without departing from the spirit of the present methods.

Method 600 begins with detect operation 602, wherein the ventilator-ECGE system detects that improper ventilation and/or oxygenation is implicated based on a comparison of sensor output to one or more threshold, as described above in method 500.

At identify parameters operation 604, the ventilator-ECGE system may identify one or more parameters that implicated improper oxygenation and/or ventilation. In some embodiments, in order to prevent unnecessary alarms, notifications, and/or recommendations, thresholds and conditions are utilized by identify parameters operation 604 to determine when improper ventilation and/or oxygenation has occurred with sufficient frequency to warrant notification of the operator. In some embodiments, the identify parameters operation 604 is performed by the manage module 126, controller 110, processor 116 and/or pneumatic system 102 that are discussed in detail above. For example, in some embodiments, improper oxygenation and/or ventilation that occurs in a breath in isolation from any other breath with improper oxygenation and/or ventilation will not be considered enough to warrant an occurrence of improper oxygenation and/or ventilation by identify parameters operation 604.

For example, the ventilator-ECGE may recognize that improper oxygenation and/or ventilation was implicated when sensor output breached one or more threshold for more than 10 milliseconds (ms) or for more than two consecutive breaths. For example, in another embodiment, the ventilator-ECGE system at detect improper treatment operation 512 determines that improper ventilation and/or oxygenation is implicated when one or more of the following sensor outputs breach a predetermined threshold: PₐCO₂, pH, PₐO₂, E_{T}CO₂, VCO₂, SpO₂, and/or etc.

The thresholds listed above are just one example list of possible conditions that could be used to indicate improper ventilation and/or oxygenation in the parameters operation 604. Any suitable list of conditions for determining the occurrence of improper ventilation and/or oxygenation may be utilized by the parameters operation 604. As may be appreciated, the ventilator-ECGE system may use information regarding parameters or sensor output that implicated improper ventilation and/or oxygenation in determining an appropriate message and/or recommendation entry of the notification. Based on the parameters or sensor output that implicated the improper ventilation and/or oxygenation, the ventilator-ECGE system during the parameters operation 604 may further identify a suggested adjustment for mitigating the improper ventilation and/or oxygenation.

At identify settings operation 606, the ventilator-ECGE system may identify one or more current ventilator and/or ECGE settings associated with the treatment of the patient. For example, current settings may have been received upon initiating treatment of the patient and may have been input by the clinician (e.g., breath type, PBW or IBW, disease conditions, PEEP, FiO₂ of sweep gas, and/or etc.). As may be appreciated, the ventilator-ECGE system may use information regarding current settings in determining an appropriate notification, such a message and/or entry. In some embodiments, the identify settings operation 606 is performed by the manage module 126, compare module 124, controller 110, processor 116 and/or pneumatic system 102 that are discussed in detail above.

At determine message operation 610, the ventilator-ECGE system may determine an appropriate message. In some embodiments, the determine message operation 610 is performed by the notification module 118, controller 110, processor 116 and/or pneumatic system 102 that are discussed in detail above. For example, the appropriate notification message may alert the clinician that improper ventilation and/or oxygenation has been implicated and/or may provide information regarding the parameter(s) or sensor output that implicated improper oxygenation and/or ventilation. For example, the appropriate notification message may alert the clinician that improper oxygenation was the results of PₐO₂, blood flow, and/or SpO₂ breaching one or more thresholds. In another example, the appropriate notification message may alert the clinician that improper ventilation was the result of PₐCO₂, pH, E_{T}CO₂, and/or VCO₂ breaching one or more thresholds. In another example, the appropriate notification message may alert the clinician that improper oxygenation and/or ventilation was implicated and was mitigated by adjusting one or more of the following parameters: sweep gas flow rate, FᵢO₂ of sweep gas, blood flow, minute ventilation, peak inspiratory pressure, tidal volume, respiratory rate, PEEP, and FᵢO₂.

For example, if improper ventilation is detected because of an E_{T}CO₂ and/or VCO₂ breached one or more thresholds, the one or more notification messages may include: “Consider increasing minute ventilation, peak inspiratory pressure, tidal volume and/or respiratory rate” or “consider increasing sweep gas flow rate.” In alternative embodiments, measured parameters such as PₐCO₂, pH, blood flow, PₐO₂, SpO₂, E_{T}CO₂, and/or VCO₂ may be utilized in the notification message and/or be the basis for the notification message.

At determine primary entry operation 612, the ventilator-ECGE system may determine an appropriate primary recommendation entry. The appropriate primary recommendation entry may provide one or more specific suggestions for mitigating improper ventilation and/or oxygenation. According to some embodiments, in determining the appropriate primary recommendation entry, the ventilator-ECGE system may take into consideration the sensor output that implicated improper ventilation and/or oxygenation. In some embodiments, the determine primary entry operation 612 is performed by the notification module 118, controller 110, processor 116 and/or pneumatic system 102 that are discussed in detail above.

According to other embodiments, in determining an appropriate primary recommendation entry the ventilator-ECGE system may take into consideration one or more of the patient's settings. For example, if the patient suffers from ARDS, the ventilator may offer one or more recommendation messages that may include: “Consider increasing the FiO2 of sweep gas” or “Consider increasing blood flow.” In another example, if the patient has been treated with the ECGE system for an extended period of time, the ventilator may offer one or more recommendation messages that may include: “Consider decreasing blood flow” or “Consider deactivating the ECGE system.” Any of the primary recommendation entries as discussed above and as displayed in Table 1 above for any condition may be utilized by method 600.

In some embodiments, at determine secondary entry operation 614, the ventilator-ECGE system also determines an appropriate secondary recommendation entry. The secondary recommendation entry may provide one or more general suggestions for mitigating improper oxygenation and/or ventilation. In some embodiments, the determine secondary entry operation 614 is performed by the notification module 118, controller 110, processor 116 and/or pneumatic system 102 that are discussed in detail above. For example, the secondary recommendation entry may include: “Consider activating an ECGE system, Consider activating a ventilator system; Consider increasing blood flow, Consider increasing FᵢO₂ of sweep gas; Consider increasing PEEP, or Consider increasing FᵢO₂.” The secondary recommendation entry may provide additional recommendations for mitigating improper oxygenation and/or ventilation. In further embodiments, the appropriate secondary recommendation entry may take into consideration the patient's current settings. That is, during ventilation with just the ventilator system, the ventilator-ECGE system in the secondary recommendation entry may suggest activating the ECGE system. As known by a person of skill in the art any notification, message, entry, and/or recommendation disclosed herein may be suitable for use as a primary and/or secondary recommendation entry.

At issue notification operation 616, a notification is issued. A notification is issued when the ventilator-ECGE system alerts the clinician via any suitable means that improper ventilation and/or oxygenation has been implicated. In some embodiments, the issue notification operation 616 is performed by the notification module 118, display module 122, controller 110, processor 116 and/or pneumatic system 102 that are discussed in detail above. For example, according to embodiments, a notification may include an appropriate notification message and an appropriate recommendation entry regarding the presence of improper oxygenation and/or ventilation. Additionally or alternatively, the notification may include an appropriate notification message, an appropriate primary recommendation entry, a smart prompt and/or an appropriate secondary recommendation entry. The notification may be displayed via any suitable means, e.g., on the ventilator-ECGE GUI and/or at a remote monitoring station, such that the clinician is alerted as to the potential presence of improper ventilation and/or oxygenation and offered additional information and/or recommendations for mitigating the improper ventilation and/or oxygenation, as described herein.

In some embodiments, a ventilator-ECGE system for issuing a notification when improper ventilation and/or oxygenation is implicated during treatment of a patient is disclosed. The ventilator-ECGE system includes: means for monitoring ventilator and/or ECGE system sensor output, means for delivering breathing gas to a patient with a ventilator system based on one or more ventilator settings; means for removing carbon dioxide from and adding a gas mixture to a bloodstream of the patient to oxygenate the bloodstream of the patient with an ECGE system based on ECGE settings, means for comparing sensor output to one or more thresholds, means for issuing a notification based on the comparison of the sensor output to the one or more thresholds.

The ventilator-ECGE system includes: means for monitoring ventilator and/or ECGE system sensor output, means for delivering breathing gas to a patient with a ventilator system based on one or more ventilator settings; means for removing carbon dioxide from and adding a gas mixture to a bloodstream of the patient to oxygenate the bloodstream of the patient with an ECGE system based on ECGE settings, means for comparing sensor output to one or more thresholds, means for adjusting the ventilator setting and/or the ECGE setting based on the comparison of the sensor output to the one or more threshold.

In some embodiments, a ventilator-ECGE system for issuing a notification when improper oxygenation and/or ventilation is implicated during treatment of a patient is disclosed. The ventilator-ECGE system includes: means for detecting improper treatment of a patient based on sensor output from a ventilator sensor and/or a ECGE sensor, means for determining an appropriate message based on the improper treatment; determining an appropriate entry based on the improper treatment, and means for issuing at least one of the appropriate messages and the appropriate entry based on the improper treatment.

In further embodiments, the means for the medical ventilator are illustrated in FIGS. 1-4 and are described in the above descriptions of FIGS. 1-4. However, the means described above for FIGS. 1-4 and illustrated in FIGS. 1-4 are but one example and are not meant to be limiting.

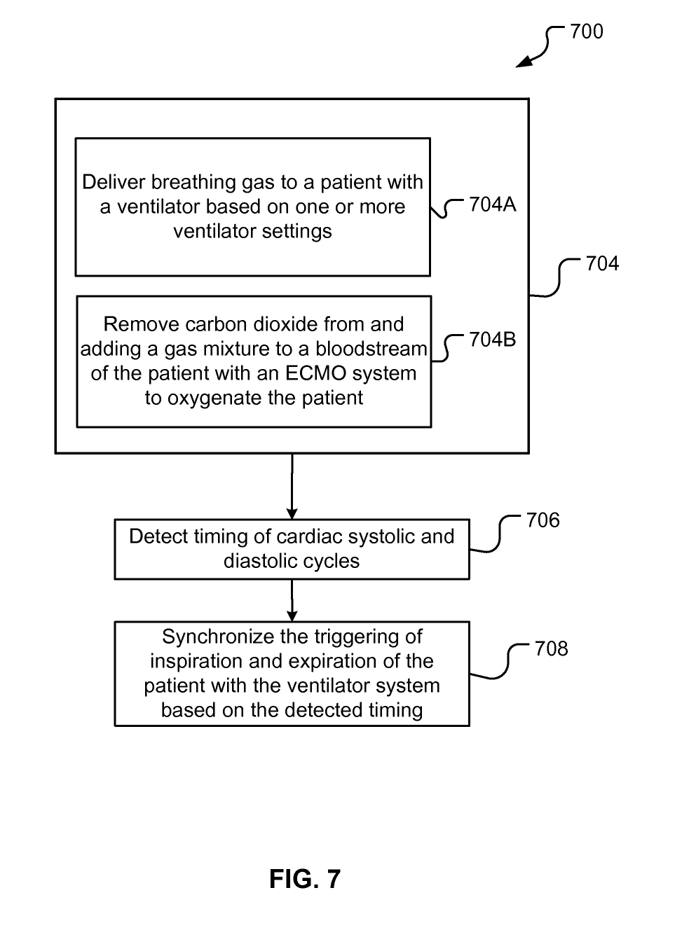

FIG. 7 illustrates an embodiment of a method 700 for optimizing synchronization of the heart and lung movements in a patient with a ventilator-ECGE system. Method 700 includes a treatment operation 704. The treatment operation is similar to the treatment operation 504 described above in method 500. At treatment operation 704, the ventilator-ECGE system provides ventilation and oxygenation to a patient. The ventilator-ECGE system during treatment operation 704 delivers breathing gas to a patient with a ventilator system based on one or more ventilator settings 704A. The ventilator-ECGE system during treatment operation 704, also, removes carbon dioxide from a patient's bloodstream and adds a gas mixture to the patient's bloodstream to oxygenate the blood stream of the patient with the ECGE system based on one or more ECGE settings 704B. In some embodiments, the ventilator-ECGE system provides ventilation and oxygenation to a patient based on operator input. In some embodiments, the ventilator-ECGE system includes an oximeter and/or capnometer.

As illustrated in FIG. 7, method 700 includes a timing operation 706. The timing operation is similar to the timing operation 506 described above in method 500. As discussed above, the settings of the ventilator can be adjusted to either load or unload cardiac output dependent upon the phasing of positive pressure and the phase of the heart beat (systolic or diastolic). Accordingly, the ventilator-ECGE system during the timing operation 506 monitors or detects the timing of cardiac systolic and diastolic cycles. In some embodiments, the ventilator-ECGE system during the timing operation 706 monitors an ECG to detect the timing of cardiac systolic and diastolic cycles of the patient's heart. In other embodiments, the ventilator-ECGE system during the timing operation 706 monitors a patient heartbeat or heart rate to detect the timing of cardiac systolic and diastolic cycles of the patient's heart. The ventilator-ECGE system during the timing operation 706 may utilize any suitable system or method for determining the heartbeat of the patient.

In some embodiments, the ventilator-ECGE system during the timing operation 706 adjusts the detected timing of the cardiac systolic and diastolic cycles to account for any appropriate delay caused by measurement, signal transfer, and/or derivations. In some embodiments, the adjustment is based on physiological and biomechanical models and signal measurement and data acquisition characteristics of the system used.

Method 700 further includes a synchronizing operation 708. The synchronizing operation 708 is similar to the synchronizing operation 508 described above in method 500. The ventilator-ECGE system during the synchronizing operation 708 synchronizes the triggering of inspiration and exhalation of the patient with the ventilator based on the detected timing to reduce a patient's cardiac load. The ventilator-ECGE system during the synchronizing operation 708 synchronizes the triggering of inspiration and/or exhalation of the patient with the ventilator based on the detected timing to reduce a patient's cardiac load by directing the ventilator to trigger an inspiration when blood is flowing out of the heart and/or to trigger an exhalation when blood is flowing into the heart. The ventilator-ECGE system during the synchronizing operation 708 synchronizes this triggering based on the detected timing of the cardiac systolic and diastolic cycles. In some embodiments, cardio respiratory synchronization may be scheduled using an adaptive phase detection and phase delay algorithm that utilizes streaming ECG waveforms (detecting the timing of cardiac systolic/diastolic cycles) and appropriate delay times to synchronize breath delivery phasing (inspiration triggering and/or expiration cycling) to optimize a desired cardio respiratory objective. The synchronization is not intended to change the ventilator's breath triggering and cycling timing in such a way that may jeopardize the respiratory needs of the patient. It will be an optimization process to determine phase adjustments within physiologically acceptable temporal limits.

In further embodiments, the ventilator-ECGE system during the synchronizing operation 708 may optimize synchronization by providing a menu of ventilator parameters such as minute volume, PEEP, target rate, oxygen mix, I:E ratio, inspiratory duration, expiratory duration, and/or etc., that may be used individually or in combinations to make adaptive ventilation adjustments to achieve better synchrony. In yet other embodiments, for passive patients, the respiratory rate for the ventilator may be derived as a proper integer ratio (fixed or user settable) of the patient's heart rate (Respiratory Rate=integer (heart rate/selected ratio)) by the ventilator-ECGE system during the synchronizing operation 708. In additional embodiments, for actively breathing patients, a form of synchronized intermittent Mandatory ventilation (SIMV) breath delivery may be used with allowable ventilator adjustments to optimize synchrony by the ventilator-ECGE system during the synchronizing operation 708.

As discussed above, an arteriovenous ECGE system utilizes the patient's heart to pump the patient's blood through the blood circuit of the ECGE system. Accordingly, the arteriovenous ECGE system increases the length and resistance through which the heart has to pump the blood volume. This extended length and increased resistance increases the cardiac load on the patient's heart. Therefore, previously, patients that are unable to sustain additional cardiac load (e.g., patients at risk for cardiac failure) were excluded from the use of arteriovenous ECGE systems. Accordingly, in some embodiments, the ventilator-ECGE system utilizes the timing operation 706 and the synchronizing operation 708 to offset at least a portion, if not all, of the increased cardiac load created by the arteriovenous ECGE system. Accordingly, ventilator-ECGE system can now be applied to patients that were previously excluded from using an arteriovenous ECGE system based on the ventilator offsetting of the increased cardiac load. However, the timing operation 706 and the synchronizing operation 708 of method 700 can be utilized by the ventilator-ECGE system to decrease the cardiac load of any patient being treated with the ventilator-ECGE system. In some embodiments, the timing operation 706 and the synchronizing operation 708 are performed by the synchronize module 128, controller 110, processor 116 and/or pneumatic system 102 that are discussed in detail above.

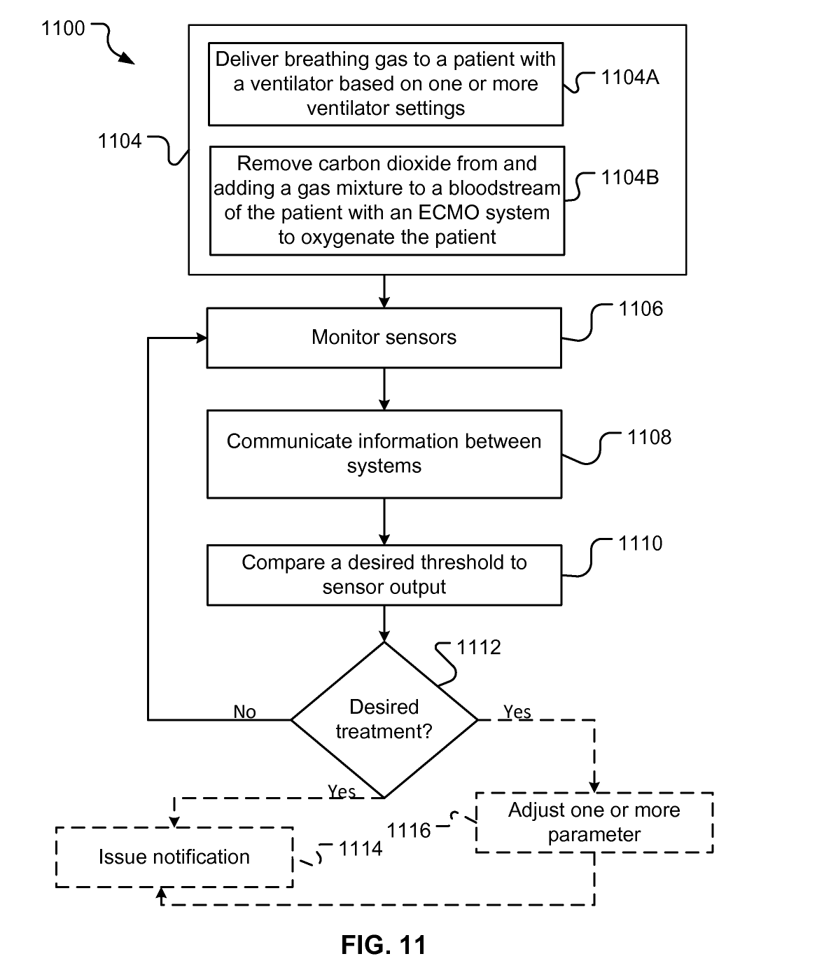

FIG. 11 illustrates an embodiment of a method 1100 for managing treatment of a patient connected to medical system. The medical system of method 1100 may be any medical system for treating a patient that includes a ventilator and an ECGE system, such as a ventilator-ECGE system. While having different meanings, the “ventilator-ECGE system” and the “medical system” may be referred to during method 1100 interchangeably. The Method 1100 includes a treatment operation 1104. The treatment operation 1104 is similar to the treatment operation 504 described above in method 500. At treatment operation 1104, the ventilator-ECGE system provides ventilation and/or oxygenation to a patient. In some embodiments, the ventilator-ECGE system during treatment operation 1104 delivers breathing gas to a patient with a ventilator system based on one or more ventilator settings 1104A. In some embodiments, the ventilator-ECGE system during treatment operation 1104, also, removes carbon dioxide from a patient's bloodstream and adds a gas mixture to the patient's bloodstream to oxygenate the blood stream of the patient with the ECGE system based on one or more ECGE settings 1104B. Accordingly, the ventilator-ECGE system during treatment operation 1104 may provide just ventilation with the ventilator, just oxygenation with the ECGE system, or a combination of overlapping or simultaneous ventilation and oxygenation by the ventilator with the ECGE system. In some embodiments, the ventilator-ECGE system during treatment operation 1104 provides ventilation and/or oxygenation to a patient based on operator input. In other embodiments, the ventilator-ECGE system during treatment operation 1104 provides ventilation and/or oxygenation to a patient based on data from a collecting data operation, such as the operation 502 described above in method 500. In some embodiments, the ventilator-ECGE system during method 1100 utilizes an oximeter and/or capnometer.

As illustrated, method 1100 includes a monitoring sensor operation 1106. The ventilator-ECGE system during monitoring sensor operation 1106 receives sensor output from the monitoring of one or more sensors. The sensors may be a ventilator sensor, an ECGE sensor, a capnometer sensor, an oximeter sensor, and/or any other suitable sensors for treatment of a patient with a medical system. In some embodiments, ventilator-ECGE system during monitoring sensor operation 1106 includes receiving derived data from a processor based on the monitored sensor output. In some embodiments, the sensor output is transmitted by sensors. In some embodiments, sensor output, such as flow rate, circuit pressure, flow pattern, inspiratory time setting (T_{I}), sweep gas flow rate, blood flow, pH, PₐCO₂, E_{T}CO₂, VCO₂, and/or etc., may be collected from the sensors, operator interface, controller, and/or processor.

Additionally, method 1100 includes a communicating operation 1108. The ventilator-ECGE system during the communicating operation 1108 communicates information between the ventilator system and the ECGE system. In some embodiments, during communicating operation 1108, the ventilator-ECGE system communicates information between the ventilator system, the ECGE system, an oximeter, and/or a capnometer. The communication may flow in one direction or in both directions. Further, the communication may include data, signals, commands, and/or instructions. Further, the communication may be wireless or wired between the systems.

Method 1100 further includes a comparing operation 1110. The ventilator-ECGE system during the comparing operation 1110 compares the sensor output (which includes output derived from sensor output) to one or more desired thresholds. In some embodiments, the ventilator-ECGE system during comparing operation 1110 compares determined trends in sensor output to the one or more desired thresholds. In other embodiments, ventilator-ECGE system during comparing operation 1110 compares sensor output from ECGE sensors to one or more desired thresholds. In further embodiments, the ventilator-ECGE system during comparing operation 1110 compares sensor output from a capnometer sensor and/or an oximeter sensor to one or more desired thresholds. In other embodiments, the ventilator-ECGE system during comparing operation 1110 compares sensor output from ventilator sensors and ECGE sensors to the same desired threshold. In further embodiments, the ventilator-ECGE system during comparing operation 1110 compares sensor output from one or more ventilator sensors to a first desired threshold and sensor output from ECGE sensors to a second desired threshold. In additional embodiments, the ventilator-ECGE system during comparing operation 1110 compares sensor output from one or more ventilator sensors to a first plurality of desired thresholds and compares sensor output from the ECGE sensors to a second plurality of desired thresholds. The first plurality of thresholds may be equivalent, overlap, or be entirely different from the second plurality of thresholds.

In some embodiments, the desired threshold is received from operator input. In other embodiments, the desired threshold is determined by the ventilator-ECGE system. In some embodiments, the ventilator-ECGE system determines the desired threshold based on predetermined settings and patient characteristics, such as patient age, patient height, patient weight, patient predicted ideal body weight, patient gender, patient disease state, ventilation mode, and/or sweep gas flow rate. This list is exemplary only and is not meant to be limiting. In some embodiments, the one or more desired thresholds are an E_{T}CO₂ level, a VCO₂ level, a SpO₂ level, a PₐCO₂ level, a pH level, a blood flow level, a PₐO₂ level, a PₐO₂/FᵢO₂, tidal volume, inspiratory pressure, and/or an OI. In other embodiments, the one or more desired threshold is a combination of two or more of the following parameters/levels: an E_{T}CO₂ level; a VCO₂ level; a SpO₂ level; a PₐCO₂ level; a pH level; a blood flow level; a PₐO₂ level; a PₐO₂/FᵢO₂; tidal volume; inspiratory pressure; and/or an OI.

As illustrated, method 1100 includes a desired treatment operation 1112. At desired treatment operation 1112 the ventilator-ECGE system determines whether the patient is receiving the desired ventilation and/or oxygenation treatment by the ventilator-ECGE system. The ventilator-ECGE system during the desired treatment operation 1112 determines if one or more setting needs to be adjusted based on information from the comparing operation 1110. In some embodiments, at detect desired treatment operation 1112 the ventilator-ECGE system determines that the patient is receiving an improper treatment when the sensor output breaches the desired threshold. In some embodiments, at detect desired treatment operation 1112 the ventilator-ECGE system determines that the patient is receiving the proper treatment when the sensor output does not breach the desired threshold. In other embodiments, at detect desired treatment operation 1112 the ventilator-ECGE system determines that the patient is not receiving the desired treatment when the sensor output breaches the desired threshold at a predetermined frequency. For example, in one embodiment, the ventilator-ECGE system at detect desired treatment operation 1112 determines that the patient is not receiving the desired treatment when a sensor output breaches the desired threshold for two or more consecutive breaths or for a predetermined time period, such as 10 ms or more.

If the ventilator-ECGE system determines that the patient is receiving the desired treatment, the ventilator-ECGE system during the desired treatment operation 1112 may return to monitoring sensor operation 1106. However, in some embodiments, the ventilator-ECGE system continuously performs the monitoring sensor operation 1106, the treatment operation 1104, comparing operation 1110, and determine desired treatment operation 1112. If the desired ventilation and/or oxygenation treatment is not being delivered to the patient, then the ventilator-ECGE system during the determine desired treatment operation 1112 may proceed to either issuing notification operation 1114 or adjusting a setting operation 1116. In some embodiments, the ventilator-ECGE system is configured to perform either or both the issuing notification operation 1114 and the adjusting a setting operation 1116. In some embodiments, the adjusting a setting operation 1116 is performed only upon user selection. In alternative embodiments, the ventilator-ECGE system is configured to perform the issuing notification operation 1114 and not the adjusting a setting operation 1116.

The thresholds listed above are just one example list of possible conditions that could be used to determine if the patient is receiving a desired ventilation and/or oxygenation during the detect desired treatment operation 1112. Any suitable list of conditions, sensor output, and/or settings for determining is the patient is receiving the desired ventilation and/or oxygenation may be utilized by the determine desired treatment operation 1112. As may be appreciated, the ventilator-ECGE system may determine whether the patient is receiving the desired ventilation and/or oxygenation at determine desired treatment operation 1112 via any suitable means. Indeed, any of the above described parameters may be evaluated according to various desired thresholds for detecting desired ventilation and/or oxygenation. Further, the disclosure regarding specific parameters as they may implicate desired treatment is not intended to be limiting. In fact, any suitable parameter or sensor output may be monitored and evaluated for detecting desired oxygenation and/or ventilation within the spirit of the present disclosure. As such, if it is detected that the patient is not receiving the desired oxygenation and/or ventilation by any suitable means, the determine desired treatment operation 1112 may proceed to the issuing notification operation 1114 and/or the adjusting a setting operation 1116.

At issuing notification operation 1114, the ventilator-ECGE may alert the clinician via any suitable means that the desired treatment (i.e., ventilation and/or oxygenation) is not being delivered to the patient. For example, according to embodiments, the ventilator-ECGE system may display a notification including a message and/or a recommendation entry regarding the desired treatment and/or the reason an undesired treatment is being delivered. According to alternative embodiments, the ventilator-ECGE system may communicate the notification, including the message and/or the recommendation entry, to a remote monitoring system communicatively coupled to the ventilator-ECGE system. According to alternative embodiments, the issued notification is any visual and/or audio notification.

According to embodiments, the notification may alert the clinician that the patient is not receiving the desired ventilation and/or oxygenation and, optionally, may provide information regarding any parameter(s) that implicated the undesirable ventilation and/or oxygenation. According to additional embodiments, the notification may provide one or more suggestions for delivering the desired oxygenation and/or ventilation. In some embodiments, the suggestions or entries are based on information from the monitoring sensor operation 1106, the comparing operation 1110, and/or the determine desired treatment operation 1112. According to further embodiments, the one or more suggestions may be based on the patient's particular settings (e.g. breath type, flow pattern, flow rate, PEEP, and/or etc.) and/or diagnosis. According to some embodiments, the clinician may access one or more parameter settings and/or display screens from the notification via a hyperlink or otherwise for addressing an undesired treatment. According to additional or alternative embodiments, a clinician may remotely access one or more parameters and/or display screens from the notification via a hyperlink or otherwise for remotely addressing the undesirable ventilation and/or oxygenation.

Additionally, the ventilator-ECGE system may generate various graphical representations of the collected and/or derived parameter data, e.g., flow waveforms, pressure waveforms, pressure-volume loops, flow-volume loops, pictorial representation of the lung, and/or etc. that are displayed during issuing notification operation 1114.

In some embodiments, method 1100 includes an adjusting a setting operation 1116. At adjusting a setting operation 1116, the ventilator-ECGE system adjusts one or more ventilator settings and/or one or more ECGE settings to maintain the desired ventilation and/or oxygenation. At adjusting a setting operation 1116, the ventilator-ECGE system adjusts one or more ventilator settings and/or one or more ECGE settings based on information from the comparing operation 1110 and/or the determine desired treatment operation 1112. In further embodiments, at adjusting a setting operation 1116, the ventilator-ECGE system adjusts one or more ventilator settings and/or one or more ECGE settings based on the comparison of the sensor output to one or more desired thresholds by the comparing operation 1110.

For example, in some embodiments, if the ventilator-ECGE system during adjusting operation 516 determines that the PₐCO₂, pH, and/or CO₂ removal rate are not within the desired threshold and need to be adjusted based on information from the comparison made by the comparing operation 1110 and/or determine desired treatment operation 1112, the ventilator-ECGE system adjusts the sweep gas flow rate to achieve the desired PₐCO₂, pH, and/or CO₂ removal rate. Although the parameters of PₐCO₂, pH, and/or CO₂ removal rate are associated with the ECGE system, the breach of these parameters can be addressed by the ventilator and/or the ECGE system. In another example, in some embodiments, if the ventilator-ECGE system during adjusting a setting operation 1116 determines that the PₐO₂ and/or blood flow rate are not within the desired threshold and need to be adjusted based on information from the comparison made by the comparing operation 1110 and/or determine desired treatment operation 1112, the ventilator-ECGE system adjusts the FᵢO₂ of the sweep gas and/or a blood flow rate of the pump, if a pump is utilized in the ECGE system, to achieve the desired PₐO₂ and/or blood flow rate. In an additional example, in some embodiments, if the ventilator-ECGE system during adjusting a setting operation 1116 determines that the E_{T}CO₂ and/or VCO₂ are not within the desired threshold and need to be adjusted based on information from the comparison made by the comparing operation 1110 and determination made by the determining a desired treatment operation 1112, the ventilator-ECGE system adjusts the minute ventilation, peak inspiration pressure, tidal volume, and/or respiratory rate to achieve the desired E_{T}CO₂ and/or VCO₂. Although the parameters of E_{T}CO₂ and/or VCO₂ are associated with the ventilator, the breach of these parameters may be addressed by the ventilator and/or ECGE system.

In another example, in some embodiments, if the ventilator-ECGE system during adjusting a setting operation 1116 determines that the SpO₂ needs to be adjusted because it is not within the desired threshold, based on information from the comparison made by the comparing operation 1110 and the determination made by the detect desired treatment operation 1112, the ventilator-ECGE system adjusts the FᵢO₂ delivered to the patient's lungs, minute volume, peak inspiratory pressure, the PEEP, FᵢO₂ concentration in the sweep gas, and/or blood flow rate to achieve the desired SpO₂. Although the SpO₂ parameter is associated with ventilation, the breach of SpO₂ may be addressed by the ventilator and/or the ECGE system. However, under some conditions the deficiency in blood oxygenation and/or ventilation is caused by physiologic etiologies that adjustments of ventilator parameters may not yield timely and/or efficient improvements. If the ventilator-ECGE system during adjusting a setting operation 1116 determines that the SpO₂ needs to be adjusted after a change in one or more of above listed ventilator parameters has been made, the ventilator-ECGE system may choose to increase the above listed ECGE parameters instead of or in combination with the above listed ventilator parameters.

The ventilator-ECGE system during adjusting a setting operation 1116 may have different set goals or multiple goals in managing the oxygenation and/or ventilation of the patient. For example, the ventilator-ECGE system during adjusting a setting operation 1116 may be set to optimize ventilation, to optimize oxygenation, and/or to protect the lungs by reducing pressure applied to lungs. Further, the ventilator-ECGE system during adjusting a setting operation 1116 may be able to achieve the same goal by adjusting different parameters or a combination of different parameters. This flexibility allows the ventilator-ECGE system during adjusting a setting operation 1116 to adjust the settings of ventilator and/or ECGE system if more or less ventilation is desirable or if more or less ECGE is desirable based on patient needs. As discussed above, a patient with ARDS may only be able to tolerate a low amount of pressure ventilation. Further, if the ECGE system has been utilized for an extended period of time, the ventilator-ECGE system during adjusting a setting operation 1116 may choose to reduce the blood flow rate in the ECGE system to prevent necrosis.

For example, if the ventilator-ECGE system during adjusting a setting operation 1116 determines that one or more setting needs to be adjusted to optimize ventilation, the ventilator-ECGE system can increase the sweep flow rate and/or increase minute ventilation (by either increasing the respiratory rate and/or the tidal volume). In another example, if the ventilator-ECGE system during adjusting a setting operation 1116 determines that one or more setting needs to be adjusted to protect the lungs, the ventilator-ECGE system can decrease tidal volume or peak inspiratory pressure for an increase in sweep flow rate. However, in this example, if the increase in sweep flow rate does not result in the desired CO₂ removal, an increase in minute ventilation can provide for additional CO₂ removal. In another example, if the ventilator-ECGE system during adjusting a setting operation 1116 determines that one or more setting needs to be adjusted to optimize oxygenation, the ventilator-ECGE system can increase blood flow rate, FᵢO₂ concentration in the sweep gas, PEEP, and/or FᵢO₂ delivered to the lungs of the patient to optimize the oxygenation of the patient.

In some embodiments, the ventilator-ECGE system during adjusting a setting operation 1116 utilizes algorithms to develop an adaptive control scheme. In some embodiments, the ventilator-ECGE system during adjusting a setting operation 1116 utilizes adaptive algorithms with online reinforced learning paradigms to develop adaptive control schemes. For example, the ventilator-ECGE system during adjusting a setting operation 1116 may make automatic adaptive adjustments to ventilator settings to optimize ventilation and/or oxygenation and may when a saturation effect is detected (no observable change caused by ventilator adjustments over a predetermined period of time) or when a declining trend in efficiency or efficacy of ventilator parameter adjustments is detected based on information from the comparing operation 1110 will make an ECGE setting adjustment.

In some embodiments, where one of the ventilator and the ECGE system are not being actively utilized, the ventilator-ECGE system during adjusting a setting operation 1116 will activate the inactive portion (the ventilator or the ECGE system) to treat the patient. In other embodiments, where both the ventilator and the ECGE system are being utilized to treat the patient, the ventilator-ECGE system during adjusting a setting operation 1116 will deactivate or shut down portions (the ventilator or the ECGE system). In some embodiments, the ventilator-ECGE system during adjusting a setting operation 1116 will activate or deactivate the appropriate system or portion.

For example, in some embodiments, the ventilator-ECGE system during adjusting a setting operation 1116 will turn off the ECGE system or recommend excluding use of the ECGE system if one or more of the following conditions occur:
if PₐO₂/FiO₂ is less than 100 for more 10 days for infants or more than 5 days for adults; and
if evidence or indications of intracranial hemorrhage (grade II or more) is present. In some embodiments, the ventilator-ECGE system during adjusting a setting operation 1116 will recommend switching from an arterial-venous ECGE system to a veno-venous or a veno-arterial ECGE system if one or more of the following conditions is detected:
evidence indicating an inability to maintain systemic perfusion,
a deterioration of cardiac function;
poor cardiac function on echocardiogram;
high serum lactate (e.g., >8 mmol/l); and
persistent metabolic acidosis.
   In other embodiments, the ventilator-ECGE system during adjusting a setting operation 1116 will turn off the ventilator system or recommend excluding use of the ventilator system if one or more of the following conditions is detected:
trending of physiologic and setting parameters indicating a risk of lung injury;
exacerbation of lung injury due to high inspiratory pressure, high PEEP, or high minute volume; and
data trends indicate an inefficacy and/or no response to changes or adjustment to ventilator settings and/or persistent oxygen dependence.

In some embodiments, in cases where even low levels of positive pressure would increase damage to the lungs, oxygenation and ventilation could be accomplished using ECGE, with minimal contribution by the ventilator. Once the lungs have had a chance to recover, the ventilator settings can be increased so as to increase the contribution of mechanical ventilation. At some point the ECGE contribution would be reduced to zero as the respiratory system recovered. The mechanical ventilator would remain connected to the patient at all times, as a means of backup in case of problems with the ECGE system (e.g., leakage within the pump).

In some embodiments, after performing the adjusting a setting operation 1116, the ventilator-ECGE system will perform the issuing notification operation 1114. According to embodiments, the ventilator-ECGE system during an issuing notification operation 1114, issues a notification to alert the clinician that the desired ventilation and/or oxygenation was not being delivered to the patient and that this was mitigated with an appropriate adjustment, and optionally, may provide information regarding any parameter(s) that breached a desired threshold.

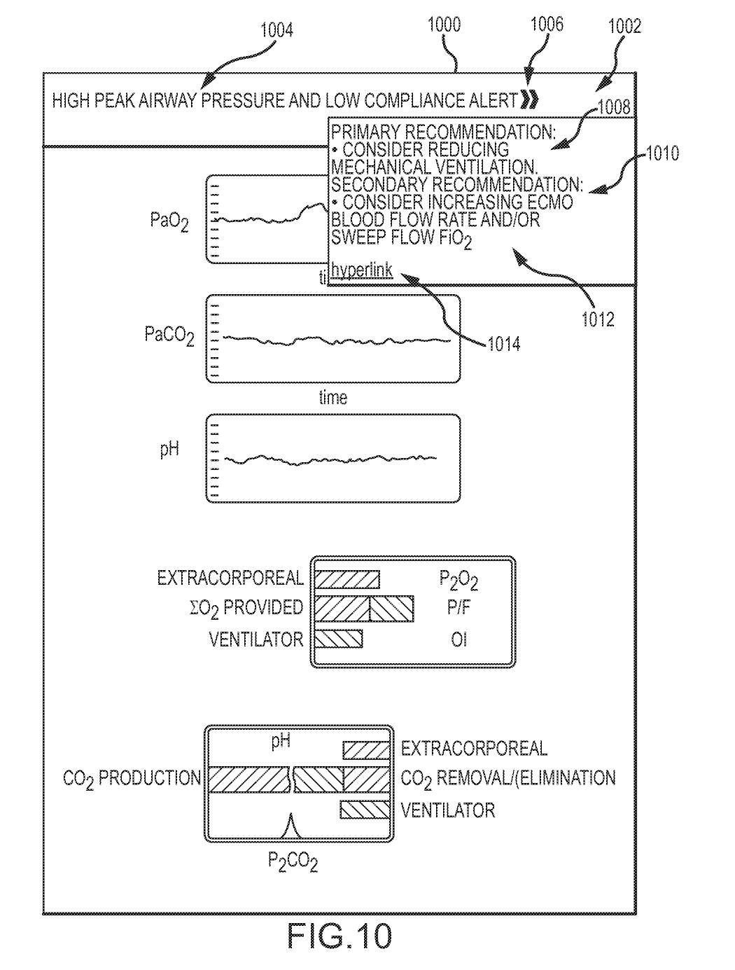

FIG. 10 is an illustration of an embodiment of a graphical user interface 1000 displaying a notification 1002.

Graphical user interface 1000 may display various monitored and/or derived data to the clinician during ventilation and/or oxygenation of a patient. In addition, graphical user interface 1000 may display various messages to the clinician (e.g., alarm messages, etc.). Specifically, graphical user interface 1000 may display notification 1002 as described herein.

According to embodiments, the ventilator may monitor and evaluate various ventilator and ECGE parameters based on one or more predetermined thresholds to detect an inadequate ventilation and/or oxygenation. As illustrated, a PₐO₂ waveform may be generated and displayed by the ventilator on graphical user interface 1000. Upon a determination that a parameter breaches a predetermined threshold, the graphical user interface 500 may display a notification 1002, e.g., a message 1004.

According to embodiments, notification 1002 may be displayed in any suitable location such that a clinician may be alerted regarding a detected patient condition, but while allowing other displays and data to be visualized substantially simultaneously. As illustrated, notification 1002 is presented as a bar or banner across an upper region of the graphical user interface 1000. However, as previously noted, notification 1002 may be displayed as a tab, icon, button, banner, bar, or any other suitable shape or form. Further, notification 1002 may be displayed in any suitable location within the graphical user interface 1000. For example, notification 1002 may be located along any border region of the graphical user interface 1000 (e.g., top, bottom, or side borders) (not shown), across an upper region (shown), or in any other suitable location. Further, as described herein, notification 1002 may be partially transparent such that displays and data may be at least partially visible behind notification 1002.

Specifically, notification 1002 may alert the clinician that improper oxygenation and/or ventilation is detected or that a parameter breached a predetermined threshold, for example by message 1004. As described herein, message 1004 may alert the clinician that the improper ventilation and/or oxygenation is detected via any suitable means, e.g., “Inadequate Oxygenation Alert,” “Inadequate Ventilation Detected,” or “Inadequate Ventilation and Oxygenation Implicated.” Notification 1002 may further include information regarding parameters that implicated the inadequate flow as illustrated in message 1004. According to alternative embodiments, in addition to the message 1004, one or more recommendation entries may be provided in an initial smart prompt banner or in a drop down menu or expanded section selected by the operator. According to other embodiments, rather than providing information regarding parameters that implicated an improper oxygenation or ventilation in the initial message, this information may be provided within an expanded portion 1012 of notification 1002.

According to embodiments, notification 1004 is expanded to provide additional information and/or recommendations to the clinician regarding a detected patient condition. For example, an expand icon 1006 may be provided within a suitable area of the notification 1004. According to embodiments, upon selection of the expand icon 1006 via any suitable means, the clinician may optionally expand the notification 1006 to acquire additional information and/or recommendations for mitigating the detected patient condition. In some embodiments the recommendations or entries are divided into a primary entry 1008 and a secondary entry 1010. According to further embodiments, notification 1002 may include links or hyperlinks 1114 to additional settings and/or display screens of the graphical user interface 1000 such that the clinician may easily and quickly mitigate and/or verify the detected condition.

As may be appreciated, the disclosed data, graphics, and notification illustrated in graphical user interface 1000 may be arranged in any suitable order or configuration such that information and alerts may be communicated to the clinician in an efficient and orderly manner. The disclosed data, graphics, and notification are not to be understood as an exclusive array, as any number of similar suitable elements may be displayed for the clinician within the spirit of the present disclosure. Further, the disclosed data, graphics, and notification are not to be understood as a necessary array, as any number of the disclosed elements may be appropriately replaced by other suitable elements without departing from the spirit of the present disclosure. The illustrated embodiment of the graphical user interface 1000 is provided as an example only, including potentially useful information and alerts that may be provided to the clinician to facilitate communication of detected improper ventilation and/or oxygenation in an orderly and informative way, as described herein.

In some embodiments, methods 700 and 1100 are performed by the ventilator-ECGE system illustrated in FIGS. 1-4 and described above. In an alternative embodiment, a computer-readable medium having computer-executable instructions for performing methods 700 and 1100 are disclosed. These methods include repeatedly performing the steps illustrated in FIG. 7 or FIG. 11 and as described in the descriptions of FIGS. 7 and 11 above.

In another embodiment, the medical system includes: means for performing each of the operations illustrated in FIG. 7 and as described above in the description of FIG. 7. In one embodiment, the means are illustrated in FIGS. 1-4 and described in the above description of FIGS. 1-4. However, the means described above for FIGS. 1-4 and illustrated in FIGS. 1-4 are but one example only and are not meant to be limiting.

In another embodiment, the medical system includes: means for performing each of the operations illustrated in FIG. 11 and as described above in the description of FIG. 11. In one embodiment, the means are illustrated in FIGS. 1-4 and described in the above description of FIGS. 1-4. However, the means described above for FIGS. 1-4 and illustrated in FIGS. 1-4 are but one example only and are not meant to be limiting.

Those skilled in the art will recognize that the methods and systems of the present disclosure may be implemented in many manners and as such are not to be limited by the foregoing exemplary embodiments and examples. In other words, functional elements being performed by a single or multiple components, in various combinations of hardware and software or firmware, and individual functions, can be distributed among software applications at either the client or server level or both. In this regard, any number of the features of the different embodiments described herein may be combined into single or multiple embodiments, and alternate embodiments having fewer than or more than all of the features herein described are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software or firmware components described herein as would be understood by those skilled in the art now and hereafter.

Numerous other changes may be made which will readily suggest themselves to those skilled in the art and which are encompassed in the spirit of the disclosure and as defined in the claims. While various embodiments have been described for purposes of this disclosure, various changes and modifications may be made which are well within the scope of the present disclosure. Numerous other changes may be made which will readily suggest themselves to those skilled in the art and which are encompassed in the spirit of the disclosure and as defined in the claims.

What is claimed is:

1. A method for managing treatment of a patient connected to a ventilator-extracorporeal membrane gas-exchanger system, the method comprising:
delivering breathing gas to a patient with a ventilator system based on one or more ventilator settings;
monitoring ventilator sensors with the ventilator system;
removing carbon dioxide from and adding a gas mixture to a bloodstream of the patient to oxygenate the bloodstream of the patient with an extracorporeal membrane gas-exchanger (ECGE) system based on one or more ECGE settings;
monitoring ECGE sensors with the ECGE system;
communicating information between the ventilator system and the ECGE system;
comparing sensor output from the ventilator sensors to a first threshold;
comparing the sensor output from the ECGE sensors to a second threshold;
adjusting at least one of the one or more ventilator settings and the one or more ECGE settings based on the comparing of the sensor output to the first threshold and the comparing of the sensor output to the second threshold.

2. The method of claim 1, wherein the first threshold is the same as the second threshold.

3. The method of claim 1, wherein the first threshold and the second threshold are received from operator input via the ventilator system;

4. The method of claim 1, wherein the first threshold is at least one of a E_{T}CO₂ level, a VCO₂ level, minute ventilation, tidal volume, peak inspiratory pressure, and a SpO₂ level and wherein the second threshold is at least one of a PₐCO₂ level, a pH level, blood flow level and a PₐO₂ level.

5. The method of claim 1, wherein the one or more ventilator settings is at least one of minute ventilation, peak inspiratory pressure, tidal volume, respiratory rate, Positive End Expiratory Pressure (PEEP), and FiO₂ and wherein the one or more ECGE settings are a sweep gas flow rate, FiO₂ of sweep gas, and blood flow.

6. The method of claim 1, wherein the adjusting step is performed by a controller on the ventilator system.

7. The method of claim 1, further comprising displaying the sensor output generated by the ventilator sensors and the ECGE sensors together on a ventilator display.

8. The method of claim 1, wherein the comparing step and the adjusting step further include one of:
determining that the sensor output from the ventilator sensors breached the first threshold and the sensor output from the ECGE sensors breached the second threshold by the comparing step and adjusting the one or more ECGE settings by the adjusting step;
determining that the sensor output from the ventilator sensors breached the first threshold and the sensor output for the ECGE sensors breached the second threshold by the comparing step and adjusting the one or more ECGE settings and adjusting the one or more ventilator settings by the adjusting step;
determining that the sensor output from the ventilator sensors breached the first threshold and the sensor output for the ECGE sensors breached the second threshold by the comparing step and adjusting the one or more ventilator settings by the adjusting step;
determining that the sensor output from the ventilator sensors breached the first threshold by the comparing step and adjusting the one or more ventilator settings by the adjusting step;
determining that the sensor output from the ventilator sensors breached the first threshold by the comparing step and adjusting the one or more ECGE settings by the adjusting step;
determining that the sensor output from the ventilator sensors breached the first threshold by the comparing step and adjusting the one or more ECGE settings and the one or more of ventilator settings by the adjusting step;
determining that the sensor output from the ECGE sensors breached the first threshold by the comparing step and adjusting the one or more ventilator settings by the adjusting step;
determining that the sensor output from the ECGE sensors breached the first threshold by the comparing step and adjusting the one or more ECGE settings by the adjusting step;
determining that the sensor output from the ECGE sensors breached the first threshold by the comparing step and adjusting the one or more ECGE settings and the one or more ventilator settings by the adjusting step; and
determining that the sensor output from the ventilator sensors did not breach the first threshold for a predetermined period and that the sensor output from the ECGE sensors did not breached the second threshold for the predetermined period by the comparing step and adjusting the one or more ECGE settings to reduce patient reliance on the ECGE system.

9. The method of claim 1, wherein the one or more ventilator settings are adjusted to increase patient reliance on the ventilator system and the one or more ECGE settings are adjusted to decrease patient reliance on the ECGE system.

10. The method of claim 1, wherein the one or more ventilator settings are adjusted to decrease patient reliance on the ventilator system and the one or more ECGE settings are adjusted to increase patient reliance on the ECGE system.

11. A ventilator-ECGE system for issuing a notification, comprising:
at least one processor; and
at least one memory, communicatively coupled to the at least one processor and containing instructions that, when executed by the at least one processor cause a controller to issue a notification, the controller including:
a manage module that detects improper treatment of a patient based on sensor output from a ventilator sensor and from an ECGE sensor, wherein the improper treatment includes detecting at least one of improper oxygenation and improper ventilation;
a notification module that determines an appropriate message based on the improper treatment and determines an appropriate entry based on the improper treatment; and
a display module that issues at least one of the appropriate message and the appropriate entry.

12. The ventilator-ECGE system of claim 11, wherein the manage module determines the sensor output that implicated the improper treatment and communicates the sensor output that implicated the improper treatment to the notification module, and
wherein the notification module determines the appropriate message based at least in part on the sensor output that implicated the improper treatment, and
wherein the appropriate message comprises an alert that improper treatment is implicated and information regarding the sensor output that implicated improper treatment.

13. The ventilator-ECGE system of claim 11, wherein the appropriate message comprises a primary message and a secondary message and is based at least in part on the sensor output that that implicated the improper treatment.

14. The ventilator-ECGE system of claim 11, wherein the appropriate message comprises one of the following entries:
increase a sweep flow rate based on at least one of the following sensor outputs: PₐCO₂, pH, E_{T}CO₂, and VCO₂;
decrease the sweep flow rate based on at least one of the following sensor outputs: PₐCO₂, pH, E_{T}CO₂, and VCO₂;
increase a FiO₂ of a sweep gas based on at least one of the following sensor outputs: PₐO₂, blood flow, and SpO₂;
decrease the FiO₂ of the sweep gas based on at least one of the following sensor outputs: PₐO₂, blood flow, and SpO₂;
increase a blood flow through an ECGE system based on at least one of the following sensor outputs: PₐO₂, blood flow, and SpO₂;
decrease the blood flow through the ECGE system based on at least one of the following sensor outputs: PₐO₂, blood flow, and SpO₂;
increase the FiO₂ based on at least one of the following sensor outputs: PₐO₂, blood flow, and SpO₂;
decrease the FiO₂ based on at least one of the following sensor outputs: PₐO₂, blood flow, and SpO₂;
increase a PEEP based on at least one of the following sensor outputs: PₐO₂, blood flow, and SpO₂;
decrease the PEEP based on at least one of the following sensor outputs: PₐO₂, blood flow, and SpO₂;
increase a peak inspiration pressure based on at least one of the following sensor outputs: PₐCO₂, pH, E_{T}CO₂, and VCO₂
decrease the peak inspiration pressure based on at least one of the following sensor outputs: PₐCO₂, pH, E_{T}CO₂, and VCO₂;
increase a tidal volume based on at least one of the following sensor outputs: PₐCO₂, pH, E_{T}CO₂, and VCO₂;
decrease the tidal volume based on at least one of the following sensor outputs: PₐCO₂, pH, E_{T}CO₂, and VCO₂;
increase a respiratory rate based on at least one of the following sensor outputs: PₐCO₂, pH, E_{T}CO₂, and VCO₂;
decrease the respiratory rate based on at least one of the following sensor outputs: PₐCO₂, pH, E_{T}CO₂, and VCO₂;
increase minute ventilation based on at least one of the following sensor outputs: PₐCO₂, pH, E_{T}CO₂, and VCO₂; and
decrease the minute ventilation based on at least one of the following sensor outputs: PₐCO₂, pH, E_{T}CO₂, and VCO₂.

15. A ventilator-ECGE system, comprising:
a ventilator system including a pressure generating system, the pressure generating system adapted to deliver breathing gas to a patient based on ventilator settings;
an ECGE system that removes carbon dioxide from a patient's bloodstream and provides a gas mixture to the patient's bloodstream to oxygenate the patient's bloodstream based on ECGE settings;
an operator interface for receiving operator input;
a plurality of sensors operatively coupled to at least one of the pressure generating system, the patient, a patient circuit, and the patient's bloodstream wherein the plurality of sensors monitor a plurality of parameters to generate sensor output;
a controller that receives the operator input and commands the pressure generating system and the ECGE system based on the operator input; and
a display module, the display module displays the generated sensor output.

16. The ventilator-ECGE system of claim 15, further comprising:
a compare module that compares the generated sensor output to at least one threshold; and
a manage module that manages treatment of the patient by adjusting at least one of the ventilator settings and the ECGE settings based on information received from the compare module, wherein the treatment of the patient include at least one of oxygenation and ventilation of the patient.

17. The ventilator-ECGE system of claim 15, further comprising:
a compare module that compares the generated sensor output to at least one threshold; and
a notification module that issues a notification based on information received from the compare module,
wherein the display module displays the notification.

18. The ventilator-ECGE system of claim 17, wherein the notification is a recommendation entry.

19. The ventilator-ECGE system of claim 18, wherein the recommendation entry is selectable.

20. The ventilator-ECGE system of claim 15, wherein the controller and the display module are part of the ventilator system.
